(19) United States
(12) Patent Application Publication
(10) Pub. No.: US 2014/0024677 A1
(43) Pub. Date: Jan. 23, 2014

(54) METHODS FOR INDUCING MITOCHONDRIAL BIOGENESIS

(71) Applicant: MUSC Foundation For Research Development, Charleston, SC (US)

(72) Inventors: Rick G. SCHNELLMANN, Charleston, SC (US); Craig C. BEESON, Mount Pleasant, SC (US); Yuri K. PETERSON, James Island, SC (US)

(73) Assignee: MUSC Foundation For Research Development, Charleston, SC (US)

(21) Appl. No.: 13/859,546

(22) Filed: Apr. 9, 2013

Related U.S. Application Data
(60) Provisional application No. 61/621,896, filed on Apr. 9, 2012.

(51) Int. Cl. A61K 31/4704; A61K 31/138; A61K 31/137

(52) U.S. Cl. CPC A61K 31/4704; A61K 31/137; A61K 31/138
USPC 514/312; 514/653; 514/651

(57) ABSTRACT

Methods and compositions for inducing mitochondrial biogenesis are provided. In some aspects, methods for the treatment of diseases such as acute kidney disease (AKI) or a muscle wasting disease by administering tomoxetine, nisoxetine, fenoterol, formoterol, or procaterol to an individual are provided.

This application claims the benefit of U.S. Provisional Patent Application No. 61/621,896, filed Apr. 9, 2012, the entirety of which is incorporated herein by reference.

This invention was made with government support under T32 CA119945-04, F32 ES020103-01, DK062028, ES012878, DK071997, GM084147, and C06 RR-015455 awarded by National Institutes of Health. The government has certain rights in the invention.

The sequence listing that is contained in the file named “MESCP0063US_ST25.txt”, which is 4 KB (as measured in Microsoft Windows®) and was created on Jun. 26, 2013, is filed herewith by electronic submission and is incorporated by reference herein.

### BACKGROUND OF THE INVENTION

1. Field of the Invention

The present invention relates generally to the fields of molecular biology and medicine. More particularly, it concerns methods and compositions for inducing mitochondrial biogenesis.

2. Description of Related Art

Mitochondrial dysfunction is associated with the etiology of multiple diseases including Alzheimer's disease, Parkinson's disease, diabetes, as well as renal, liver and myocardial injury (Hagen et al., 2002; Baloyannis, 2006; Civitarese and Ravussin, 2008; Seo et al., 2010; Tran et al., 2011). Mitochondrial dysfunction is also a common cause and consequence of ischemia/reperfusion, trauma, and drug/toxicant-induced organ injury. In ischemic/reperfusion and lipopolysaccharide induced acute kidney injury (AKI), the loss of mitochondrial function results in oxidative stress and persistent energy depletion (Funk et al., 2010; Tran et al., 2011). Mitochondrial damage may hinder critical energy-dependent repair mechanisms and lead to irreversible cell injury, limiting restoration of organ function. Thus, the development of therapies to promote mitochondrial biogenesis (MB) has the potential to treat multiple pathologies and restore organ function after injury.

Mitochondria are constantly being renewed through the processes of biogenesis, fusion, fission, and mitophagy (Seo et al., 2010). MB occurs under homeostatic conditions, and can be induced as an adaptive response initiated by cells to meet energetic demands resulting from injury, genetic, metabolic, and dietary events, thereby impacting health and disease (Medeiros, 2008). MB is a complex process requiring the coordination of both nuclear and mitochondrial DNA (mtDNA) that encode for mitochondrial proteins. A primary regulator of MB is the nuclear transcriptional coactivator peroxisome proliferator-activated receptor gamma coactivator 1-α (PGC-1α). PGC-1α interacts with cAMP response element-binding protein and nuclear respiratory factors 1 and 2 to regulate the transcription of multiple genes (Wu et al., 2006). PGC-1α provides a direct link between external physiological stimuli and the regulation of MB, and is inducible both physiologically and pharmacologically.

There are a limited number of chemicals known to induce MB. The Spiegelman group conducted a high-throughput screen that examined the effects of 3,000 compounds in skeletal muscle cells and 82 compounds (2.5%) increased PGC-1α mRNA (Arany et al., 2008). More recently, isoflavones, a 5HT₂ agonist (1-(2,5-dimethoxy-4-iodophenyl)-2-aminopropane hydrochloride; DOI), and a SIRT1 activator (N-[2-[3-(piperazin-1-ylmethyl)imidazo[2,1-b][1,3]thiazol-6-yl]phenyl]quinoxaline-2-carboxamide; SRT1720) can MB in renal proximal tubular cells (Rasbach and Schnellmann, 2008; Funk et al., 2010; Rasbach et al., 2010). Furthermore, the inventors determined that stimulation of MB after the initiation of cellular injury accelerates cell repair and regeneration. For example, after t-butylhydroperoxide (TBHP) induced oxidant injury, exposure of primary renal proximal tubule cells (RPTC) to DOI, SRT1720 or over expression of PGC1-α accelerated recovery of mitochondrial and ATP-dependent cellular functions (Rasbach and Schnellmann, 2007; Funk et al., 2010; Rasbach et al., 2010). Therefore, targeted stimulation of MB may be a valuable approach in the development of new therapies for the treatment of injury and disease characterized by mitochondrial impairment (Funk et al., 2010).

Acute kidney injury (AKI) is a clinical disorder characterized by a rapid decrease in kidney function and subsequent retention of nitrogenous waste products, metabolic acids, and increased potassium and phosphate concentrations. AKI is prevalent in approximately 60% of patients during intensive care admission and has a mortality rate of up to 90% in critically ill and aged patients. In addition, AKI is costly to treat and has become a significant financial burden on the healthcare system. Current treatments are limited to mechanical support by dialysis. Historically, the vast majority of research efforts for AKI has focused on pretreatments, and is less clinically relevant as AKI presents as an unpredictable acute onset. Taken together the high mortality rates, financial burden, and limitations in treatment demonstrate a significant clinical need for discovery of novel approaches to therapeutics that promote recovery of renal function following AKI.

### SUMMARY OF THE INVENTION

The present invention overcomes limitations in the prior art by providing new compounds and methods for inducing mitochondrial biogenesis in a subject, e.g., a patient with a mitochondrial dysfunction or disease. In certain aspects, specific β-2 adrenergic receptor agonists and norepinepherine reuptake inhibitors have been identified that can induce mitochondrial biogenesis. In various embodiments, mitochondrial biogenesis may be induced in vitro or in vivo. Specific compounds have been identified which may be used to treat muscle wasting or acute kidney injury (AKI).

An aspect of the present invention relates to a method of inducing mitochondrial biogenesis in a subject, comprising administering to a subject, identified as in need of increased mitochondrial biogenesis, a pharmacologically effective dose of compound, wherein the compound is a beta-2 adrenergic receptor agonist or a norepinepherine reuptake inhibitor. The beta 2 adrenergic receptor agonist may be tomoxetine, nisoxetine, fenoterol, formoterol, or procaterol. In some embodiments, the method may comprise administering to the subject a pharmacologically effective dose of tomoxetine, nisoxetine, fenoterol, formoterol, or procaterol. The compound may be a beta-2 adrenergic receptor agonist. The beta-2 adrenergic receptor agonist may be fenoterol, formoterol, terbutaline, ritodrine, cyclopentylbutanephrine, metaproteranol, procaterol, nisoxetine, or tomoxetine. In some embodiments, the beta-2 adrenergic receptor agonist is not formoterol. In some embodiments, the compound is a norepinepherine reuptake inhibitor. The norepinepherine reuptake inhibitor may be nisoxetine or tomoxetine. The compound may be a compound from Table 1, such as, e.g., CB_{—}3 6431725 or CB_{—}2 5144525. The compound may be administered to the subject orally, intravenously, intramuscularly, intraperitoneally, topically, or via inhalation. The compound may be comprised in a pharmaceutically acceptable formulation.

In some embodiments, the subject is a human. In some embodiments, the subject has been identified as having deficient mitochondrial biogenesis by a muscle biopsy test, a metabolic test, or a genetic test. For example, the blood test may comprise, e.g., evaluation of basic chemistries, a complete blood count, analysis of lactate and/or pyruvate, analysis of amino acids, ammonia levels, creatinine kinase (CPK) levels, plasma acylcarnitine analysis, or analysis of urine organic acids. The muscle biopsy test may comprise evaluation of liver tissue respirometry, muscle pathology, oxidative phosphorylation enzymology, protein chemistry, or COQ10 levels. The genetic testing may evaluate sequencing mitochondrial DNA or nuclear DNA. The subject may have a mitochondrial injury. The mitochondrial injury may result from neurodegeneration, a heart disease, heart attack, a stroke, renal dysfunction, type 2 diabetes, a central nervous system disorder, Alzheimer's disease, Parkinson's disease, Huntington's disease, ischemia/reperfusion, trauma, a drug/toxicant-induced organ injury, a traumatic brain injury (TBI), chronic traumatic encephalopathy (CTE), or an acute kidney injury (AKI). The subject has a mitochondrial disease. For example, the mitochondrial disease may be Leber's hereditary optic neuropathy, diabetes mellitus, a mental disorder or disease, Leigh's disease, mitochondrial encephalomyopathy, lactic acidosis, mitochondrial neurogastrointestinal encephalomyopathy (MNGIE), Myoclonic Epilepsy with Ragged Red Fibers (MERRF), “Neuropathy, ataxia, retinitis pigmentosa, and ptosis” (NARP), Wolff-Parkinson-White syndrome, or stroke-like episodes (MELAS), or a muscle wasting disease.

Another aspect of the present invention relates to a method of treating an acute kidney injury in an individual comprising administering to the individual a pharmacologically effective amount of tomoxetine, nisoxetine, fenoterol, formoterol, or procaterol. In some embodiments, a pharmacologically effective amount of tomoxetine, nisoxetine, fenoterol, or procaterol is administered to the subject. The subject may be a human.

Yet another aspect of the present invention relates to a method of treating muscle wasting or a muscle wasting disease in an individual comprising administering to the individual a pharmacologically effective amount of tomoxetine, nisoxetine, fenoterol, or procaterol. In some embodiments, the muscle wasting disease is cachexia (e.g., as a result of cancer, increased age, spinal injury, etc.), muscle atrophy resulting from cancer (cancer cachexia), wasting disease, or muscle atrophy resulting from a spinal cord injury or neurological injury or disease. The cachexia may result from a chronic illness such as type I diabetes, multiple sclerosis, HIV, or cancer. The subject may be a human.

In some embodiments, tomoxetine((3R)-N-methyl-3-(2-methylphenoxy)-3-phenylpropan-1-amine; (R)-N-methyl-3-phenyl-3-(o-tolyloxy)propan-1-amine; also called Atomoxetine) may be administered orally, e.g., to a human subject at a dosage of about 10-100 mg/day to promote mitochondrial biogenesis. In some embodiments, 20-60 mg/day of tomoxetine may be administered orally to a human subject to promote mitochondrial biogenesis or treat a disease as described herein. In some embodiments, a dosage of 0.25-2 mg/kg/day, or 0.5-1.2 mg/kg/day may be administered to an individual to promote mitochondrial biogenesis or treat a disease as described herein. In some embodiments, concentrations of tomoxetine may be used to promote mitochondrial biogenesis that are lower than the dosages of tomoxetine that are used to treat ADHD. For example, less than about 0.25 mg/kg/day tomoxetine, from about 0.05 mg/kg/day to less than about 0.25 mg/kg/day, from about 0.1 mg/kg/day to less than about 0.25 mg/kg/day, about 0.1-0.24 mg/kg/day, about 0.05-0.24 mg/kg/day, about 0.05-0.20 mg/kg/day, about 0.05-0.15 mg/kg/day, or 0.05-0.1 mg/kg/day, or any range derivable therein, may be used to induce mitochondrial biogenesis in a subject, such as a human patient.

Nisoxetine ((RS)-3-(2-methoxyphenoxy)-N-methyl-3-phenylpropan-1-amine) may be administered to a subject, such as a human subject to promote mitochondrial biogenesis. For example, a dose of about 0.05-2 mg/kg, or about 0.1 mg/kg, may be used to promote mitochondrial biogenesis or treat a disease as described herein.

In some embodiments, fenoterol ((RR,SS)-5-(1-hydroxy-2-{[2-(4-hydroxyphenyl)-1-methylethyl]amino}ethyl)benzene-1,3-diol) may be administered to a subject, such as a human subject, to promote mitochondrial biogenesis or treat a disease as described herein. In some embodiments, fenoterol is delivered to a subject via aerosol delivery or orally. In some embodiments, 0.007-0.035 mg/kg may be administered to a human subject about every 6 hours via inhalation. 0.028-0.14 mg/kg/day may be administered to an individual via an aerosol delivery. A dosage of about 300-600 mcg/day may be administered to an individual. In some embodiments, about 100-200 mcg may be administered to a subject orally about every 8 hours. In some embodiments, about 0.01 mg/kg/day may be administered to a subject.

In some embodiments, procaterol ((±)-(1R,2S)-rel-8-Hydroxy-5-[1-hydroxy-2-(isopropylamino)butyl]-quinolin-2(1H)-one) may be administered to a subject, such as a human subject, to promote mitochondrial biogenesis or treat a disease as described herein. In some embodiments, 0.25-125 mcg/day, or 0.5-100 mcg/day of procaterol may be administered to an individual orally. For pediatric patients, a dose of about 0.625-2.5 ug/kg may be used. Procaterol may be administered, in various embodiments, orally or via inhalation. In some embodiments, the amount of procaterol administered to a subject to promote mitochondrial biogenesis may be lower than the dose that would be used clinically to treat asthma. For example, in some embodiments, 0.007-0.035 mg/kg may be administered to a human subject about every 6 hours via inhalation. 0.028-0.14 mg/kg/day may be administered to an individual via an aerosol delivery. A dosage of about 300-600 mcg/day may be administered to an individual. In some embodiments, about 100-200 mcg may be administered to a subject orally about every 8 hours. In some embodiments, about 0.01 mg/kg/day may be administered to a subject.

Formoterol (rac-(R,R)—N-[2-hydroxy-5-[1-hydroxy-2-[1-(4-methoxyphenyl)propan-2-ylamino]ethyl]phenyl]formamide) may be administered to a subject, such as a human subject, to promote mitochondrial biogenesis or treat a disease as described herein. The formoterol may be administered orally or via inhalation. In some embodiments, about 12-24 mcg/day may be administered to an individual, e.g., as an inhalation powder. In some embodiments, about 20-40 μg/2 mL of formoterol may be administered via aerosol once or twice per day. In some embodiments, the amount of formotorol administered to a subject to promote mitochondrial biogenesis may be lower than the dose that would be used clinically to treat asthma or chronic obstructive pulmonary disease. For example, in some embodiments, 0.007-0.035 mg/kg may be administered to a human subject about every 6 hours via inhalation. 0.028-0.14 mg/kg/day may be administered to an individual via an aerosol delivery. A dosage of about 300-600 mcg/day may be administered to an individual. In some embodiments, about 100-200 mcg may be administered to a subject orally about every 8 hours. In some embodiments, about 0.01 mg/kg/day may be administered to a subject.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

As used herein the specification, “a” or “an” may mean one or more. As used herein in the claim(s), when used in conjunction with the word “comprising”, the words “a” or “an” may mean one or more than one.

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or ” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Formoterol exposure increases uncoupled oxygen consumption rate and mtDNA copy number in RPTC and AFC. FIG. 1A, RPTC exposed to formoterol (β₂-AR agonist) for 24 h exhibit a significant increase in FCCP-OCR. Exposure to isoproterenol (non-specific β-AR agonist) and BRL 37344 (β₃-AR agonist) did not have any significant effects. FIG. 1B, AFC exposed to formoterol for 24 h exhibit a significant increase in FCCP-OCR. Exposure to isoproterenol and BRL 37344 did not have any significant effects. FIG. 1C, RPTC exposed to the non-specific β-AR agonists dopamine, epinephrine, and norepinephrine for 24 h did not exhibit any significant increases in FCCP-OCR. FIG. 1D, RPTC exposed to 30 nM formoterol for 24 h showed a 2.5 fold increase in mtDNA copy number relative to DMSO controls. Data is represented a mean±s.e.m. of four biological replicates (*P<0.05).

FIGS. 2A-C. Formoterol exposure induced the expression of mitochondrial genes and mtDNA copy number in the kidney cortex of CB57BL/6 mice. FIG. 2A, mRNA expression in the kidney cortex of CB57BL/6 mice 24 h after a single i.p. injection with 100 mg/kg formoterol. Mitochondrial regulatory protein PGC-1α was induced 2.5-fold. Mitochondrially-encoded genes ATP6, ND1, and ND6 were induced 2.5, 3.5 and 2.5-fold respectively. Nuclear-encoded mitochondrial gene NDUFB8 was induced 2-fold. FIG. 2B, mRNA expression in the kidney cortex of CB57BL/6 mice 72 hr after daily repeated i.p. injections with 100 mg/kg formoterol. PGC-1α, ND1, NDUFB8 induced 1.75, 2, and 2-fold respectively. FIG. 2C, mtDNA copy number significantly increased in kidney cortex 2-fold 72 h after daily repeated i.p. injection with 100 mg/kg formoterol. Values indicate fold change relative to DMSO controls. Data is represented a mean±s.e.m. of three—six biological replicates (*P<0.05).

FIGS. 3A-C. Formoterol exposure induced the expression of mitochondrial genes and mtDNA copy number in the heart of CB57BL/6 mice. FIG. 3A, mRNA expression in the heart of CB57BL/6 mice 24 h after a single i.p. injection with 100 mg/kg formoterol. Mitochondrial regulatory protein PGC-1α was induced 3-fold. Mitochondrially-encoded mitochondrial genes ATP6, ND1, and ND6 were induced 1.5, 2 and 2-fold respectively. Nuclear-encoded mitochondrial gene NDUFB8 was induced 2-fold. FIG. 3B, mRNA expression in the kidney cortex of CB57BL/6 mice 72 hr after daily repeated i.p. injections with 100 mg/kg formoterol. PGC-1α, ND1, and NDUFB8 were induced 2, 4 and 2-fold respectively. FIG. 3C, mtDNA copy number significantly increased in kidney cortex 2 and 2.5-fold 24 h and 72 h after daily repeated i.p. injection with 100 mg/kg formoterol. Values indicate fold change relative to DMSO controls. Data is represented a mean±s.e.m. of three to six biological replicates (*P<0.05).

FIGS. 4A-C. Cheminformatic analysis of formoterol identified two compounds that induce MB in RPTC, nisoxetine and tomoxetine. FIG. 4A, RPTC exposed to formoterol, nisoxetine and tomoxetine (10-300 nM) for 24 h exhibit a significant increases in FCCP-OCR. FIG. 4B, pharmacophore based on alignment of formoterol, nisoxetine, and tomoxetine. Formoterol, nisoxetine, and tomoxetine aligned with superimposed chemical features. F1 is a proton acceptor, F2 and F3 are hydrophobic, F4 is a mixed feature with a cationic and proton donor, and F5 and F6 are mixed features with aromatic or hydrophobic characteristics. F1 and F4 were marked as essential while requiring that at least 5 features matched the model. FIG. 4C, formoterol aligned with superimposed pharmacophore. Values indicate a percent of fold change relative to DMSO controls. Data is represented a mean±s.e.m. of four biological replicates (*P<0.05).

FIGS. 5A-B. The effects of formoterol, nisoxetine, and tomoxetine are inhibited by β₂ antagonism. FIG. 5A, pre-exposure to the β-AR antagonist propranolol (5 nM) inhibited the effects of 30 nM formoterol, nisoxetine and tomoxetine on FCCP-OCR in RPTC. FIG. 5B, pre-exposure to the β₂-AR inverse agonist ICI 118,551 (3 and 10 nM) inhibited the effects of 30 nM formoterol, nisoxetine and tomoxetine on FCCP-OCR in RPTC. Values indicate a percent of fold change relative to DMSO controls. Data is represented a mean±s.e.m. of four biological replicates (*P<0.05).

FIGS. 6A-C. Formoterol pharmacophore identified two ChemBridge compounds that induce MB in RPTC. FIG. 6A, RPTC exposed to ChemBridge compounds 2-3 (10-300 nM) for 24 h exhibit a significant increase in FCCP-OCR. RPTC exposed to ChemBridge compound 1 showed no change in FCCP-OCR. FIG. 6B, refined pharmacophore based on alignment of formoterol, nisoxetine, and tomoxetine, CB2 and CB3. In this alignment, F1 is a proton acceptor, F2 through F3 are hydrophobic, F4 is a mixed feature with a proton donor and a cationic or proton acceptor, and F5 and F6 are mixed features with aromatic or hydrophobic characteristics. F7 is a unique hydrophobic feature found in nisoxetine, tomoxetine and CB2. F1, F2, F4, F5, and F6 are essential features. FIG. 6C, nisoxetine aligned with superimposed pharmacophore. Values indicate a percent of fold change relative to DMSO controls. Data is represented a mean±s.e.m. of four biological replicates.

FIG. 7. Generalized chemotype of MB stimulating β₂-AR agonists and similar compounds. All compounds are in reference to the conserved phenethylamine core. Brackets represent the similarity clusters from FIG. 4.

FIG. 8. β₂-AR agonist and similar compounds induce concentration-responsive increases in FCCP-uncoupled OCR after 24 h. Values indicate a percent of fold change relative to DMSO controls. Data is represented a mean±s.e.m., N=4.

FIG. 9. β₂-AR agonist and similar compounds (10-3000 nM) induce partial (left column) or no MB (right column) in RPTC after 24 h. Values indicate a percent of fold change relative to DMSO controls. Data is represented a mean±s.e.m., N=4.

FIG. 10. Chemical clustering and reported pK_{d} do not correlate with mitochondrial biogenic activity. MAACS keyed chemical fingerprints were used to cluster compounds based on molecular similarity as measured by Tanimoto Coefficient. The chemogram was generated using Chemmine single linkage hierarchical clustering (http://chemmine.ucr.edu/). Three major clusters were identified and numbered within the chemogram. The MB heat map indicates full (dark grey), partial (light grey), and inactive (white) biogenic compounds as determined by RPTC OCR. The pK_{d} heat map indicates the reported affinity for each ligand to the β₂-AR with black being high affinity and light grey being low affinity. Tomoxetine and nisoxetine have not been reported to bind to β₂-AR. Cluster 4 represents an inactive cluster containing compounds with diminishing β₂-AR selectivity and low affinity.

FIGS. 11A-B Pharmacophore model based on alignment of Cluster 1: procaterol, formoterol and fenoterol. Procaterol, formoterol and fenoterol were flexibly aligned to superimposed chemical features. F1 is aromatic, F2 is a proton donor, F3 is a proton donor and acceptor, F5 is a cationic proton donor, and F4, F6, and F7 are hydrophobic.

FIGS. 12A-C. Pharmacophore model based on alignment of Cluster 2: nisoxetine and tomoxetine. FIG. 12A, Chemical graphs of nisoxetine and tomoxetine. FIG. 12B, Nisoxetine and tomoxetine were flexibly aligned to superimposed chemical features. F1 is a hydrophobic proton acceptor, F3 is a proton acceptor, F2 and F4 are aromatic, F5 and F7 are hydrophobic, and F6 is a cationic proton donor. This model reflects a complete coverage of the essential features within the compounds. FIG. 12C, Pharmacophore overlay of cluster 1 pharmacophore with cluster 2 pharmacophore.

FIGS. 13A-C. FIGS. 13A-B, Pharmacophore model based on alignment of Cluster 3: CB2 and CB3. CB2 and CB3 were flexibly aligned to superimposed chemical features. F1 and F3 are hydrophobic, F2 is a cationic proton donor, F4 is a proton acceptor, and F5 is aromatic. The CB 5 point pharmacophore model was used to probe the Chembridge DiverSet. FIG. 13C, RPTC exposed to CB3(10-3000 nM, #6431725) and CB2 for 24 h exhibit significant and concentration-responsive increases in FCCP-uncoupled OCR. Values indicate a percent of fold change relative to DMSO controls. Data is represented a mean±s.e.m. of four biological replicates.

FIGS. 14A-C. Cheminformatic based virtual screening and experimental validation identified novel biogenic agents. FIG. 14A, The Chembridge DiverSet conformer library was searched using the CB pharmacophore model and the rank order of RMSD fit is presented for compounds that matched all of five features. FIG. 14B, The compounds identified by pharmacophore searching were predicted for activity using 2D binary QSAR model. FIG. 14C, The 48 compounds that had a QSAR activity score of >0.5 and a pharmacophore match of <0.3 RMSD were tested for biogenesis in the respirometric assay. Validation yielded five positive compounds (black bars) and one toxic compound. Biogenesis was considered positive over an OCR of 1.1 and lines were added to the graph to depict the activity threshold. Values indicate a percent of fold change relative to DMSO controls. Data is represented a mean±s.e.m. of eight biological replicates.

FIG. 15. Development of a consensus pharmacophore model. Three 3D Pharmacophore models based on alignment of previously clustered compounds were rigidly aligned to superimpose and unify chemical features between chemical clusters. Cluster 1 model, Cluster 2 model, and Cluster 3 model were used. An overlay of the three models with cluster 1, cluster 2, and cluster 3 was generated. Features were combined into a consensus model with compounds overlaid. The final consensus pharmacophore model is shown. F1 and F4 are aromatic, F5 is a proton donor, F2 is a hydrophobic proton acceptor, and F3 and F7 are mixed acceptor/donor, and F6 and F8 are hydrophobic.

FIGS. 16A-F. Predicted poses between MB biased ligands with the β₂-AR. Using the consensus pharmacophore from FIG. 10, conformers of selected ligands were interacted with the β₂-AR Xray structure and ligand-receptor poses were generated using energy minimization. FIG. 16A, Full scale image of the β₂-AR. FIG. 16B, Zoom of the consensus pharmacophore overlaid with alprenolol. FIGS. 16C-F, Energy minimized poses of select agonists based on placement derived from the consensus pharmacophore. The figure is colored from light grey to dark grey from amino to carboxy terminus. The view in FIGS. 16C-F is from the perspective of helix 1-2 (which are not shown in FIGS. 16C-F). Amino acids are shown. Ligands are shown and bonds are shown as dotted lines.

FIGS. 17A-F. Predicted interactions between MB biased ligands with the β₂-AR. Using the consensus pharmacophore from FIG. 10, conformers of selected ligands were interacted with the β₂-AR Xray structure and ligand-receptor interaction maps generated using MOE. Arrows indicate hydrogen bonding from donor to acceptor. Hydrophobic interactions included H-arene and π-π interactions depicted in green.

FIG. 18. Tomoxetine promotes healing of muscle wasting.

FIG. 19. Formoterol administration reduces serum creatinine after I/R injury.

FIG. 20. Formoterol administration after I/R injury restored expression of KIM-1.

FIG. 21. Effect of formoterol on renal cortical morphology after I/R injury.

FIGS. 22A-E. Effect of Formoterol on expression of PGC-1α and ETC components after I/R injury.

FIGS. 23A-B. Treatment with vehicle after I/R was significantly decreased compared to all other groups, indicative of decreased mitochondrial function. FIG. 23A, Relative state 2 respiration. FIG. 23B, Relative state 3 respiration. These results of respiration are expressed as the means±sem and are relative values (% of control). b, P<0.05, n=7 versus Sham+Veh and IR+Form (one-way ANOVA followed by Fisher least-significant difference test for multicomparison).

### DESCRIPTION OF ILLUSTRATIVE EMBODIMENTS

As described herein, specific compounds have been identified as being particularly effective for promoting mitochondrial biogenesis. The stimulation of mitochondrial biogenesis (MB) via cell surface G-protein coupled receptors is a promising strategy for cell repair and regeneration. As shown in the below examples, the specificity and quantitative structure rationale of a panel of β₂-AR agonists was evaluated for possible effects on MB. β₂-AR agonists elicit three distinct phenotypes: full MB, partial MB, and non-MB. Full MB compounds represent two chemical scaffolds but three distinct chemical clusters. Clusters were subjected to pharmacophore and 2D-QSAR modeling and identified unique features conserved amongst full MB compounds and distinct features unique to MB. A novel cluster was further investigated via a combined virtual screening and experimental validation approach leading to the identification of five novel MB agents. Using informatics, the three pharmacophore models were coalesced into a consensus pharmacophore, elucidating the spatial and chemical features required to stimulate MB. Via superposition of the consensus model within the β₂-AR x-ray structure theonists binding modes were used to evaluate possible MB-specific interactions. Additional studies were conducted that have shown these compounds to be particularly effective in treating diseases including muscle wasting diseases and acute kidney disease (AKI).

β-AR receptor agonists are approved therapeutic agents for the management of asthma and other diseases. The β₂-AR has been successfully targeted for drug discovery using a ligand-based approach, resulting in the creation of multiple receptor-specific drugs (Wishart et al., 2006). Recently, Kolb et al. (2009), used the X-ray structure of the β₂-AR to conduct a structure-based screen of nearly one million commercially available molecules. Utilizing the β₂-AR crystallized in complex with the inverse agonist carazozol, the researchers identified 25 novel compounds that fit the receptor, and six of the compounds were confirmed as inverse agonists. However the therapeutic potential of β₂-AR agonists as inducers of mitochondrial biogenesis (MB) has not fully been explored (Ortega and Peters, 2010).

In the below examples the inventors use a phenotypic approach recently developed by Beeson et al. (2010), to examine the potential of β₂-AR agonism to induce MB in both the kidney and the heart. The Seahorse Biosciences extracellular flux analyzer was used to evaluate the effects of β₂-AR agonists on maximal mitochondrial respiration in primary cultures of renal proximal tubules and cardiomyocytes, two cells highly dependent on aerobic metabolism. In vivo and ligand-based in silico studies were then employed to integrate the effects of β₂-AR agonists on mitochondrial function and gene expression, with detailed analysis of chemical structure. These data were used to develop a discrete pharmacophore model capable of predicting novel compounds with MB properties.

While recent studies show that inducing mitochondrial biogenesis (MB) stimulates cell repair and regeneration, only a limited number of chemicals are known to induce MB. As shown in the examples below, to examine the impact of the β-adrenergic signaling pathway on mitochondrial biogenesis, primary renal proximal tubule cells (RPTC) and adult feline cardiomyocytes (AFC) were exposed for 24 hours to multiple β-adrenoceptor (β-AR) agonists: isoproterenol (non-selective β-AR agonist), BRL 37344 (selective β3-AR agonist), and formoterol (selective β2-AR agonist). The Seahorse Biosciences Extracellular Flux (XF) analyzer was used to quantify FCCP-uncoupled oxygen consumption rate (OCR), a marker of maximal electron transport chain activity. Isoproterinol and BRL 37244 did not significantly alter mitochondrial respiration at any of the concentrations examined. Formoterol exposure resulted in significant increases in both FCCP-uncoupled OCR and mitochondrial DNA (mtDNA) copy number. The effect of formoterol on OCR in RPTC was inhibited by the β-AR antagonist propranolol. Mice exposed to formoterol for 24 or 72 h exhibited increases in kidney and heart mtDNA copy number, PGC-1α, and multiple genes involved in the mitochondrial electron transport chain (ATP6, ND1, ND6, and NDUFB8). Cheminformatic modeling, virtual chemical library screening, and experimental validation identified two chemically similar compounds from the Sigma Library of Pharmacologically Active Compounds (tomoxetine and nisoxetine), and two from the ChemBridge DIVERSet™ that caused significant increases in mitochondrial respiratory capacity. These data provide compelling evidence for the use of β₂-AR ligands for therapeutic MB.

Abbreviations Used: AFC, adult feline cardiomyocytes; ATP6, ATP synthase F0 subunit 6 of transmembrane F-type ATP synthase; β-AR, β-adrenoceptor; DOI, 1-(2,5-dimethoxy-4-iodophenyl)-2-aminopropane hydrochloride; FCCP, carbonylcyanide p-trifluoromethoxyphenylhydrazone; i.p., intraperitoneal injection; LOPAC, Library of Pharmacologically Active Compounds; MB, mitochondrial biogenesis; mtDNA, mitochondrial DNA; ND1, NADH dehydrogenase subunit 1; ND6, NADH dehydrogenase subunit 6; NDUFB8, NADH dehydrogenase[ubiquinone] 1 beta subcomplex subunit 8; OCR, oxygen consumption rate; PGC-1α, peroxisome proliferator-activated receptor gamma coactivator 1-α; RPTC, renal proximal tubule cells; SMILES, Simplified Molecular Input Line Entry Specification; SRT1720, N-[2-[3-(piperazin-1-ylmethyl)imidazo[2,1-b][1,3]thiazol-6-yl]phenyl]quinoxaline-2-carboxamide; TBHP, t-butylhydroperoxide; TC, tanimoto coefficient; XF-96, Seahorse Biosciences extracellular flux analyzer.

The mitochondrion is an intricate organelle, with components derived from both the nuclear and mitochondrial genomes, whose activity must be carefully coupled to cellular metabolism and signaling (Wagner et al., 2008). Mitochondrial health is essential for cell and organ function. Multiple diseases are characterized by mitochondrial impairment including diabetes, Alzheimer's disease, Parkinson's disease and diet-induced obesity. Ischemia/reperfusion injury of the kidney, heart and liver all show deleterious consequences on mitochondrial function. Therefore, the development of novel pharmaceuticals that induce MB may have broad usage in diverse diseases. Despite the potential for treating disorders characterized by mitochondrial impairment, very few therapies target the mitochondria to promote its function (Funk et al., 2010). Likewise, there are no current databases matching mitochondrial biogenic activity to chemical pharmacophores or specific chemotypes.

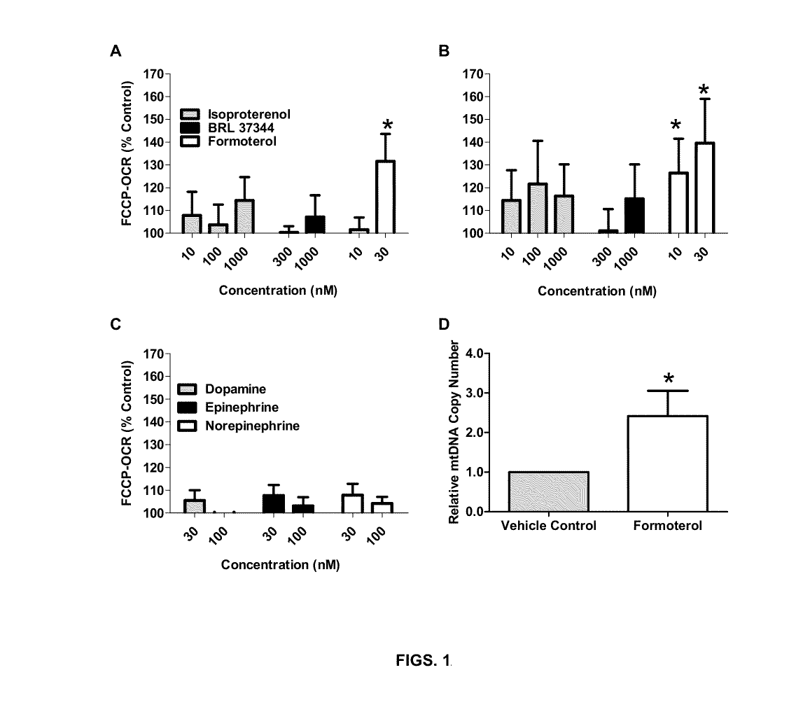

Very few studies have explored MB as a therapeutic target and those studies that have focused on skeletal muscle and assay systems using surrogate markers of MB (e.g. PGC-1α mRNA). Surrogate markers may suffer from false-positives and negatives, and the use of cell lines that are highly glycolytic with limited aerobic capacity may not be sensitive to MB. The use of primary cultures of primary RPTC grown under improved conditions resolves the cell line issue and the use of a phenotypic respirometric assay minimizes the limitations of surrogate markers. As shown below, formoterol, a potent and selective agonist for the β₂-AR, stimulates MB in both RPTC and AFC within 24 h of a 30 nM exposure (FIG. 1). Neither selective β₃-AR agonist (BRL 37344) nor non-selective β-AR agonists (dopamine, epinephrine, norepinephrine, or isoproterenol) stimulate MB in RPTC. These data suggest functional selectivity of the β₂-AR Additionally the ability of formoterol to increase FCCP-OCR was inhibited by the β-AR antagonist propranolol and the β₂-AR inverse agonist ICI 115,881. These data provide further evidence that specific stimulation of the β₂-AR induces MB in vitro.

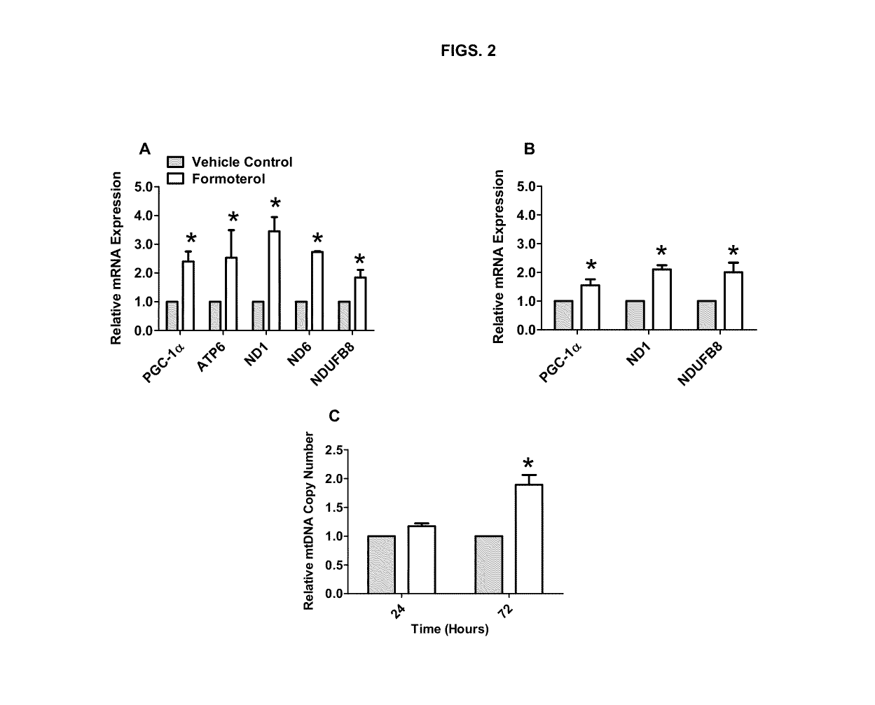

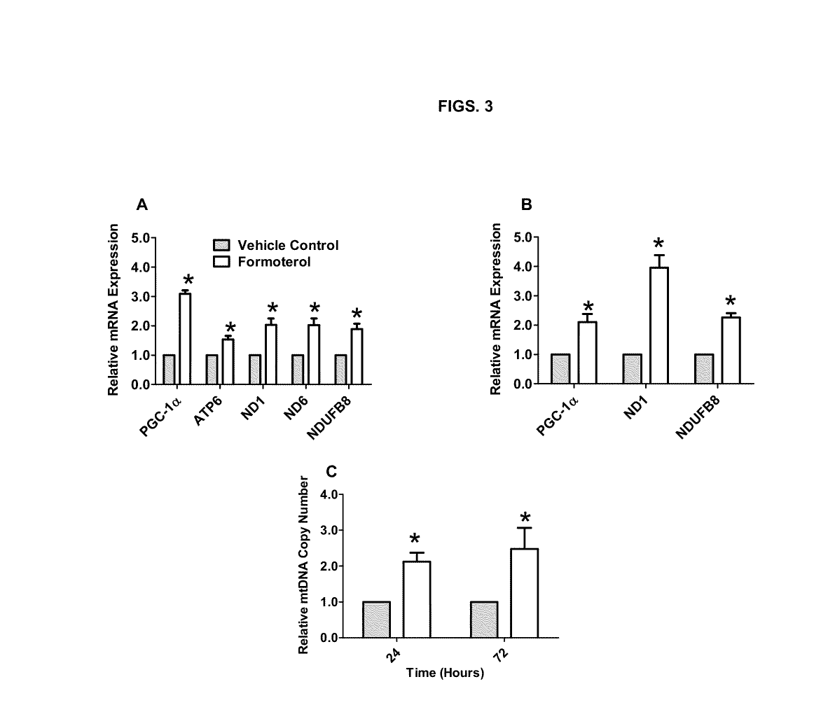

Researchers have showed that exercise-induced increases in PGC-1α mRNA in skeletal muscle, is blocked by the β₂-AR inhibitor ICI 118,551 and β-AR inhibitor propranolol (Miura et al., 2007; Sutherland et al., 2009). Additionally PGC-1α in skeletal muscle 24 h increased after injection of formoterol (100 μg/kg) (Pearen et al., 2009). Here, the same dose of formoterol induced MB in the kidney and heart of male mice as indicated by increased mRNA expression of multiple mitochondrial proteins that are both nuclear and mitochondrially-encoded (NDUFB8, AT6, ND1, ND6) (FIG. 2-3). Furthermore, PGC1-α, the master regulator of MB, was induced both 24 and 72 h after formoterol exposure. Using the aforementioned markers of MB, these data suggest that formoterol does induce MB in vivo and in both the kidney and the heart.

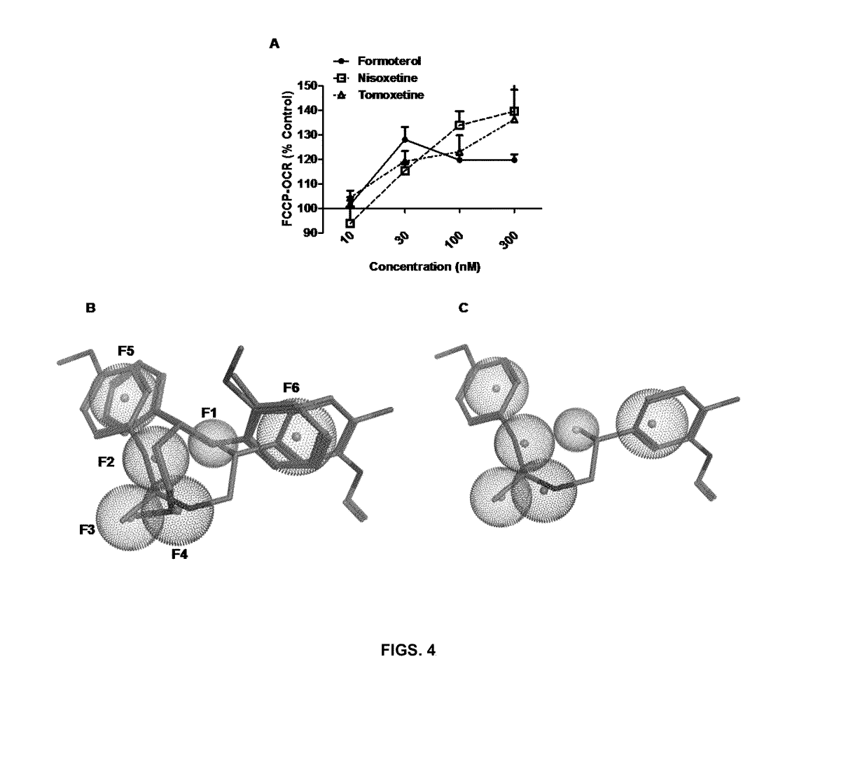

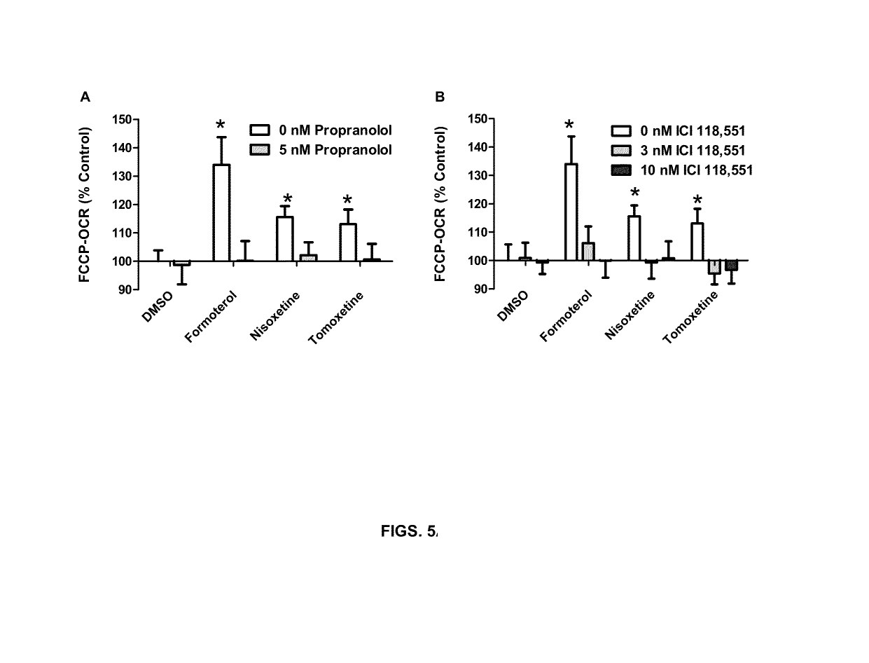

Currently there are no known pharmacophores associated with MB. The chemical structure of formoterol was utilized to search the Sigma LOPAC of 1280 compounds to find current pharmacological compounds that were similar in structure to formoterol and therefore may induce MB. 29 compounds were identified and were tested for their ability to increase the FCCP-OCR in RPTC after a 24 h 5 μM exposure. Tomoxetine and nisoxetine were identified as potent inducers (30-300 nM) of MB. Nisoxetine is reported to be a potent and selective inhibitor of noradrenaline uptake with little or no affinity for a range of other similar neurotransmitter receptors (Wong and Bymaster, 1976; Cheetham et al., 1996; Mochizucki, 2004). Tomoxetine is also a potent and selective noradrenaline re-uptake inhibitor, and is clinically used as an antidepressant (Zerbe et al., 1985). Currently there are no data suggesting a role for either nisoxetine or tomoxetine in inducing MB. Interestingly these compounds were aligned with formoterol and a preliminary pharmacophore was developed with six features common between all three compounds (FIG. 4B). Additionally, propranolol and ICI 118,551 blocked the MB effects of both compounds (FIG. 5). These data provide strong evidence that nisoxteine and tomoxetine are agonists at the β₂ AR receptor.

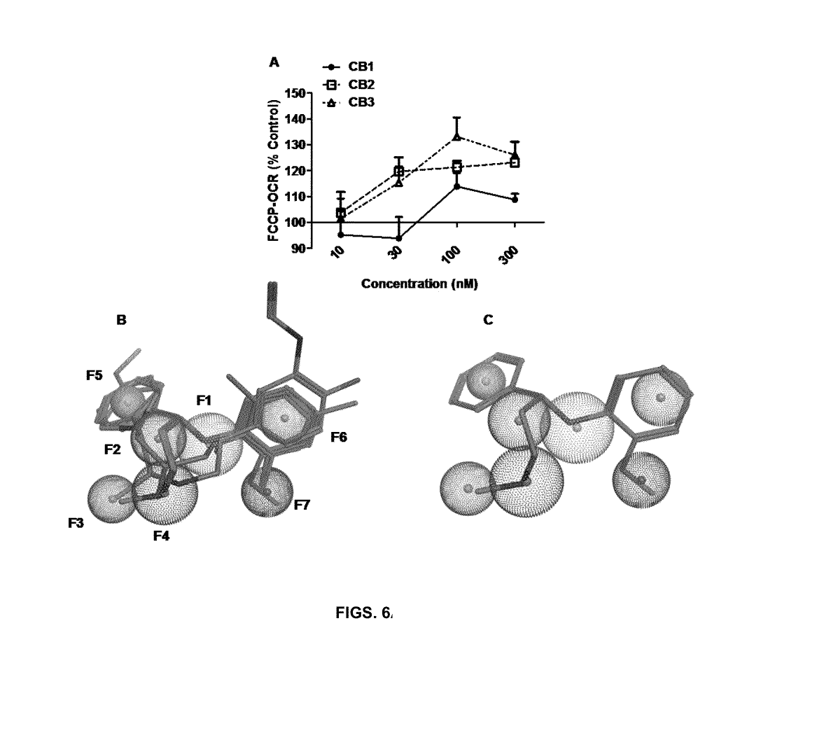

In addition to finding pharmacologically active compounds with MB activity, the formoterol-based pharmacophore was utilized to probe diverse chemical space. The pharmacophore designed from formoterol, nisoxetine and tomoxetine was used to screen the 50,000 small molecule ChemBridge DIVERSet™. The in silico screen identified only 16 compounds that matched all six chemical features that defined the pharmacophore. When tested utilizing RPTC, three of the compounds (19% accuracy in prediction, enrichment factor of 6.9) increased FCCP-OCR 24 h after a 5 μM exposure. A concentration response curve for the three compounds revealed CB2 and CB3 increased MB at 30 nM while CB1 had no effect at concentrations below 5 μM. The positive compounds were aligned, resulting in the development of a refined pharmacophore with six features present within 100% of the compounds (FIG. 6B). Negative data are not included as no clear consensus to exclusion space was reached. This improved pharmacophore can be used to search libraries such as the World Drug index to find other compounds that match the pharmacophore and induce MB. In addition, future studies will attempt to identify a broader range of novel agonists for β₂-AR mediated MB by utilizing a structure-based approach of the β₂-AR as outlined by Kolb et al (2009).

There is an abundance of evidence showing that exercise induces MB in skeletal muscle, and recent data suggests that β-AR agonism is required for exercise-mediated alterations in mitochondrial function (Miura et al., 2007; Higashida et al., 2008; Sutherland et al., 2009; Little et al., 2011). However, the potential use for β₂-AR agonists for the treatment of mitochondrial dysfunction and injury has not yet been explored. Although evidence suggests that chronic exposure to formoterol does decrease oxidative capacity in the heart, acute exposures to β₂-AR agonists do have the potential to activate MB after insult with limited detrimental effects (Ortega and Peters, 2010; Léger et al., 2011). Agonists for the β₂-AR are promising therapeutics for the treatment of mitochondrially-related organ dysfunction found in diseases including diabetes, as well as to promote recovery after injury, including acute kidney injury. The inventors have identified a role of formoterol in the induction of MB in both the kidney and the heart. Additionally the chemical structure of formoterol was utilized to identify four new mitochondrial biogenic compounds. Future studies will focus on the potential of formoterol exposure to promote recovery of kidney and heart mitochondrial function in both disease and injury models.

Table 1 below shows Formoterol pharmacophore extraction of the 1280 compound Sigma Library of Pharmacologically Active Compounds (LOPAC). Chemical fingerprints defined within the MOE software package were used to cluster compounds based on molecular similarity as measured from the Tanimoto Coefficient (TC). Analysis of the data utilizing a TC of 60% identified 23 compounds out of 1280 (or 1.8%) compounds from LOPAC that matched formoterol based on chemical similarity. RPTC were treated with the 23 identified LOPAC compounds (5 μM) for 24 hr and examined for changes in FCCP-OCR. This assay identified two compounds, nisoxetine and tomoxetine, that increase FCCP-OCR 15% above vehicle control. Fold change in FCCP-OCR relative to DMSO controls appears in the bottom left of each compound. CAS numbers appear to the bottom right.

Table 2 below shows the 6-point formoterol pharmacophore model was used to search the ChemBridge DIVERSet™ for similar compounds. This library contains 50,080 unique drug-like compounds that cover pharmacophore diversity. These compounds were used to create a 1,420,467 entry conformer library. An in silico search identified 16 compounds (or 0.03%) containing all six chemical features with a RMSD<1 Å to the pharmacophore model for all features. RPTC were exposed to the 16 identified ChemBridge compounds (5 μM) for 24 hr and examined for changes in FCCP-OCR. This assay identified three novel compounds (CB1-3) that increase FCCP-OCR 15% above the vehicle control. Fold change in FCCP-OCR relative to DMSO controls appears in the bottom left of each compound. CAS numbers appear to the bottom right.

Table 3 below shows Formoterol, nisoxetine, tomoxetine, and (CB 2) ChemBridge #5144525. The original pharmacophore was built using formoterol, nisoxetine and tomoxetine. CB 1-3 were found using the original pharmacophore from a search of the ChemBridge DIVERSet™ 50,000 small molecule conformational library. ChemBridge compounds 2 and 3 were confirmed biogenic; however, CB 2 was the only compound to add features to the pharmacophore model.

TABLE 1

| | | Average uncoupled | |
|---|---|---|---|
| Structure | Name | OCR ratio | CAS number |
| 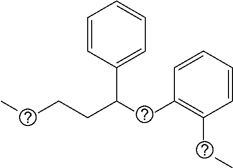 [SMILES: CCCCC(Cc1ccccc1CC)c1ccccc1] | Nisoxetine | 1.2 ± 0.08 | 57754-86-6 |
| 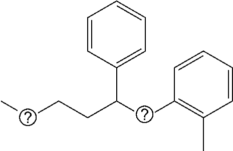 [SMILES: CCCCC(Cc1ccccc1C)c1ccccc1] | Tomoxetine | 1.14 ± 0.07 | 83015-26-3 |
| 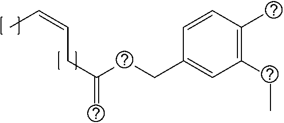 | Olvanil | 1.12 ± 0.08 | 58493-49-5 |
| 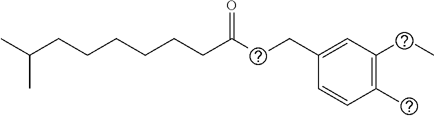 [SMILES: CCc1cc(CCC(=O)CCCCCCC(C)C)ccc1C] | Dihydrocapsaicin | 1.12 ± 0.1 | 19408-84-5 |
| 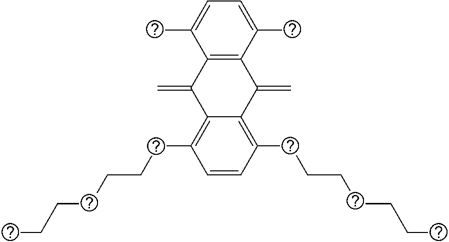 [SMILES: C=c1c2c(C)ccc(C)c2c(=C)c2c(CCCCCCC)ccc(CCCCCCC)c12] | Mitoxantrone | 1.1 ± 0.06 | 65271-80-9 |
| 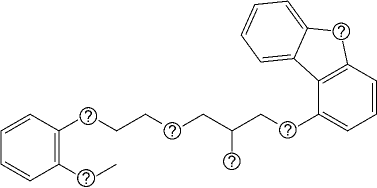 [SMILES: CCc1ccccc1CCCCCC(C)CCc1cccc2c1-c1ccccc1C2] | Carvedilol | 1.09 ± 0.03 | 72956-09-3 |
| 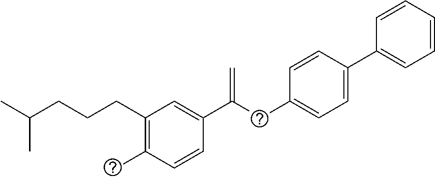 [SMILES: C=C(Cc1ccc(-c2ccccc2)cc1)c1ccc(C)c(CCCC(C)C)c1] | GR 55562 | 1.09 ± 0.09 | 159533-26-3 |
| 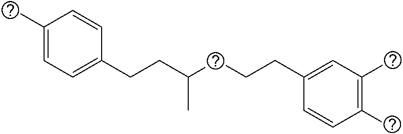 | Dobutamine | 1.09 ± 0.14 | 49745-95-1 |
| 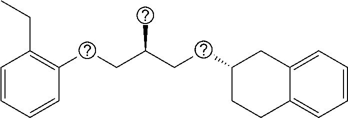 [SMILES: CCc1ccccc1CC[C@@H](C)CC[C@H]1CCc2ccccc2C1] | SR 59230A | 1.09 ± 0.07 | 174689-39-5 |
| 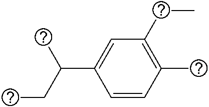 | (±)-Normetanephrine | 1.06 ± 0.03 | 1011-74-1 |
| 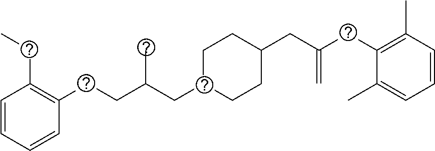 [SMILES: C=C(Cc1c(C)cccc1C)CC1CCC(CC(C)CCc2ccccc2CC)CC1] | Ranolazine | 1.03 ± 0.03 | 95635-56-6 |
| 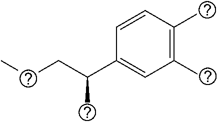 | (−)-Epinephrine | 1.03 ± 0.1 | 51-42-3 |
| 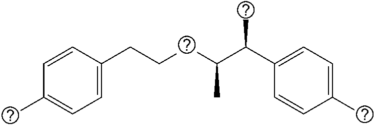 | Ritodrine | 1.02 ± 0.09 | 23239-51-2 |
| 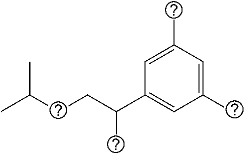 | Metaproterenol | 1.02 ± 0.06 | 586-06-1 |
| 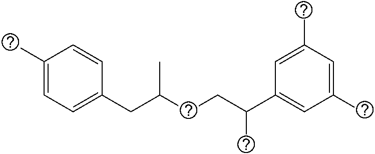 | Fenoterol | 0.98 ± 0.04 | 12/3/1944 |
| 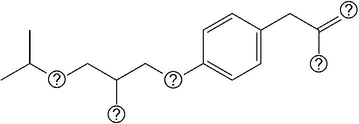 [SMILES: C=C(C)Cc1ccc(CCC(C)CCC(C)C)cc1] | S(−)-Atenolol | 0.98 ± 0.12 | 93379-54-5 |
| 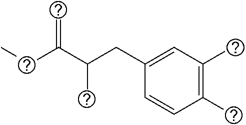 | L-3,4- Dihydroxyphenylalanine methyl ester | 0.95 ± 0.07 | 7101-51-1 |
| 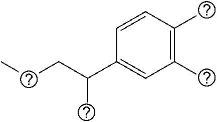 | (±)-Epinephrine | 0.92 ± 0.03 | 329-63-5 |
| 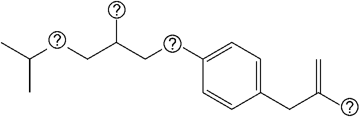 [SMILES: C=C(C)Cc1ccc(CCC(C)CCC(C)C)cc1] | (±)-Atenolol | 0.92 ± 0.15 | 29122-68-7 |
| 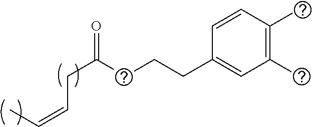 | N-Oleoyldopamine | 0.9 ± 0.09 | 105955-11-1 |
| 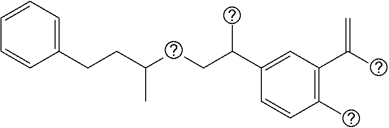 [SMILES: C=C(C)c1cc(C(C)CCC(C)CCc2ccccc2)ccc1C] | Labetalol | 0.86 ± 0.11 | 32780-64-6 |
| 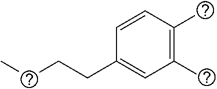 | N-Methyldopamine | 0.82 ± 0.13 | 62-32-8 |
| 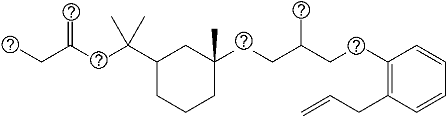 [SMILES: C=CCc1ccccc1CCC(C)CC[C@]1(C)CCCC(C(C)(C)CC(=C)CC)C1] | Bromoacetyl alprenolol menthane | 0.68 ± 0.2 | 958697-99-9 |

TABLE 2

| | | Average Drug | |
|---|---|---|---|
| | | FCCP OCR/ | |
| Structure | ID | Control FCCP OCR | CAS numbers |
| 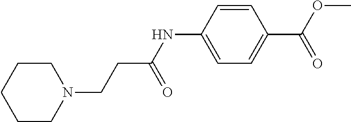 [SMILES: COC(=O)c1ccc(NC(=O)CCN2CCCCC2)cc1] | CB_3-6431725 | 1.22 ± 0.04 | 398470-83-2 |
| 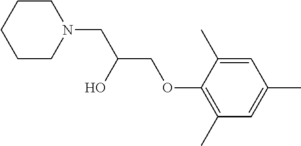 [SMILES: Cc1cc(C)c(OCC(O)CN2CCCCC2)c(C)c1] | CB_2-5144525 | 1.22 ± 0.08 | 300852-45-3 |
| 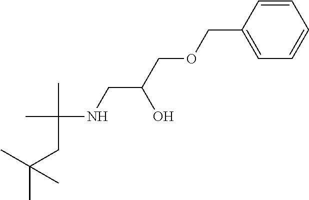 [SMILES: CC(C)(C)CC(C)(C)NCC(O)COCc1ccccc1] | CB_1-5144510 | 1.19 ± 0.08 | 765894-01-7 |
| 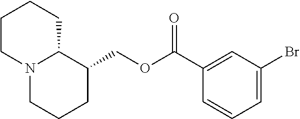 [SMILES: O=C(OC[C@@H]1CCCN2CCCC[C@H]12)c1cccc(Br)c1] | 5402373 | 1.14 ± 0.05 | 329720-38-9 |
| 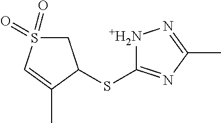 | 6384391 | 1.14 ± 0.08 | 443652-15-1 |
| 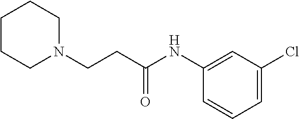 [SMILES: O=C(CCN1CCCCC1)Nc1cccc(Cl)c1] | 6434119 | 1.09 ± 0.03 | 339228-37-4 |
| 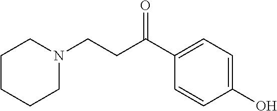 [SMILES: O=C(CCN1CCCCC1)c1ccc(O)cc1] | 6433097 | 1.09 ± 0.06 | 42528-76-7 |
| 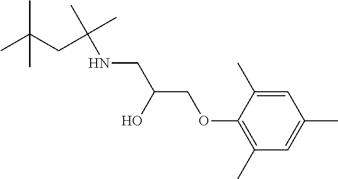 [SMILES: Cc1cc(C)c(OCC(O)CNC(C)(C)CC(C)(C)C)c(C)c1] | 5144511 | 1.08 ± 0.06 | 314027-94-6 |
| 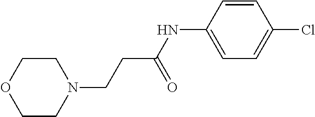 [SMILES: O=C(CCN1CCOCC1)Nc1ccc(Cl)cc1] | 6435349 | 1.01 ± 0.05 | 313962-89-9 |
| 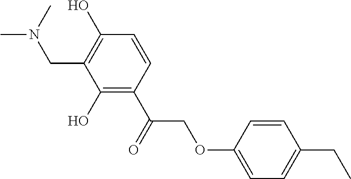 [SMILES: CCc1ccc(OCC(=O)c2ccc(O)c(CN(C)C)c2O)cc1] | 6632718 | 1 ± 0.05 | 377074-32-3 |
| 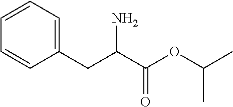 | 5108436 | 1 ± 0.11 | 81084-82-4 |
| 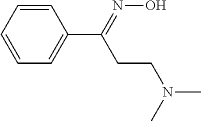 | 5533967 | 0.99 ± 0.07 | 309251-09-0 |
| 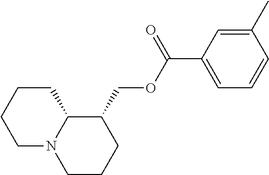 [SMILES: Cc1cccc(C(=O)OC[C@@H]2CCCN3CCCC[C@H]23)c1] | 5402374 | 0.94 ± 0.04 | 1209049-21-7 |
| 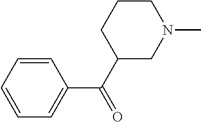 | 5244600 | 0.92 ± 0.1 | 70029-33-3 |
| 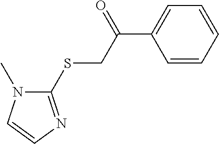 [SMILES: Cn1ccnc1SCC(=O)c1ccccc1] | 6111462 | 0.89 ± 0.04 | 263869-50-7 |
| 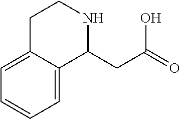 [SMILES: O=C(O)CC1NCCc2ccccc21] | 7562564 | 0.85 ± 0.04 | 105400-81-5 |

TABLE 3

| Structure | Molecule Name | CAS number |
|---|---|---|
| 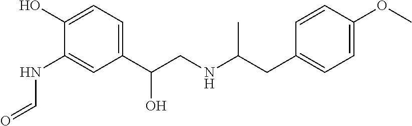 [SMILES: COc1ccc(CC(C)NCC(O)c2ccc(O)c(NC=O)c2)cc1] | Formoterol hemifumarate | 73573-87-2 |
| 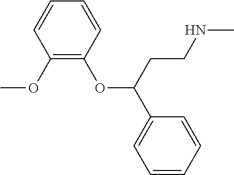 | Nisoxetine | 57754-86-6 |
| 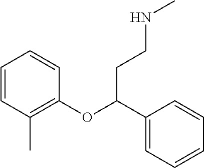 [SMILES: CNCCC(Oc1ccccc1C)c1ccccc1] | Tomoxetine | 83015-26-3 |
| 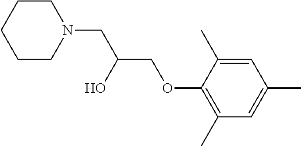 [SMILES: Cc1cc(C)c(OCC(O)CN2CCCCC2)c(C)c1] | CB_2 | 463929-24-0 |

SUPPLEMENTARY TABLE 1
Primer sequences used for qRT-PCR experiments.

| Gene | Sequence 5′-3′ | |
|---|---|---|
| APOB | FW: CGT CGG CTC CAG CAT TCT | (SEQ ID NO: 1) |
| | REV: TCA CCA GTC ATT TCT GCC TTT G | (SEQ ID NO: 2) |
| ATP6 | FW: TCC TGC CTC ACT CAT TCA CAC CAA | (SEQ ID NO: 3) |
| | REV: AGT GCA GCG CCT CCA ATA AGA TGT | (SEQ ID NO: 4) |
| D-Loop | D1: CCC AAG CAT ATA AGC TAG TAC | (SEQ ID NO: 5) |
| | D2: ATA TAA GTC ATA TTT TGG GAA CTA C | (SEQ ID NO: 6) |
| ND1 | FW: TAG AAC GCA AAA TCT TAG GG | (SEQ ID NO: 7) |
| | REV: TGC TAG TGT GAG TGA TAG GG | (SEQ ID NO: 8) |
| ND6 | FW: ACT GCG ATG GCA ACT GAG GAG TAT | (SEQ ID NO: 9) |
| | REV: ACC ATA ACT ATA CAA CGC CGC CAC | (SEQ ID NO: 10) |
| NDUFB8 | FW: ACC CAA TCC AAC CGC CTT CA | (SEQ ID NO: 11) |
| | REV: CTA GGA CCC CAG AGG AAC GC | (SEQ ID NO: 12) |
| PGC-1α | FW: AGG AAA TCC GAG CTG AGC TGA ACA | (SEQ ID NO: 13) |
| | REV: GCA AGA AGG CGA CAC ATC GAA CAA | (SEQ ID NO: 14) |
| Tubulin | FW: CTC TCT GTC GAT TAC GGC AAG | (SEQ ID NO: 15) |
| | REV: TGG TGA GGA TGG AGT TGT AGG | (SEQ ID NO: 16) |

### I. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Chemistry

All solvents and reagents, unless otherwise stated, were supplied by Sigma-Aldrich Chemical Co. Ltd. and were used as supplied.

(±)-(R*,R*)-[4-[2-[[2-(3-Chlorophenyl)-2-hydroxyethyl]amino]propyl]phenoxy]acetic acid sodium hydrate (BRL 37344), (R*,R*)—N-[2-Hydroxy-5-[1-hydroxy-2-[[2-(4-methoxyphenyl)-1-methylethyl]amino]ethyl]phenyl]formamide fumarate (formoterol furmarate dihydrate), and (R)-3,4-Dihydroxy-α-(isopropylaminomethyl)benzyl alcohol hydrochloride (isoproterenol hydrochloride) were purchased from Sigma-Aldrich Chemical Co. Ltd. and were confirmed to be 98% pure by HPLC. The Library of Pharmacologically Active Compounds (LOPAC) was purchased from Sigma-Aldrich Chemical Co. Ltd. and the compounds were confirmed to be 95% pure by HPLC. The ChemBridge DIVERSet™ 50,000 compound library was purchased from ChemBridge Corporation and the compounds were confirmed to be 95% pure by HPLC.

Biological Evaluation of Compounds

Isolation of proximal tubules. Female New Zealand white rabbits (1.5-2.0 kg) were purchased from Myrtle's Rabbitry (Thompson Station, Tenn., USA). Rabbit renal tubules were isolated using the iron oxide perfusion method as described in Nowak et al. (1995). The resulting proximal tubules were plated on 100-mm tissue culture-grade plastic Petri dishes constantly swirled on an orbital shaker at 80 rpm. The culture medium was a 50:50 mixture of Dulbecco's modified Eagle's essential medium and Ham's F12 (lacking glucose, phenol red, and sodium pyruvate; Gibco BRL) supplemented with 5 μg/ml human transferrin, 5 ng/ml selenium, 50 nM hydrocortisone, and 10 nM bovine insulin. After three days the de-differentiated cells were trypsinized and re-plated onto XF-96 polystyrene cell culture microplates at a concentration of 18,000 cells/well and maintained in a 37° C. incubator for two days prior to experimentation (Beeson et al., 2010).

Isolation of primary adult feline cardiomyocytes. Adult feline cardiomyocytes were isolated to 95% purity as published previously (Mann et al., 1989). In the present study, glass-bottom dishes (MatTek) coated with laminin were used for culturing cardiocytes in Piper's medium, which was prepared in M199 cell culture medium (Invitrogen) containing the following additional ingredients: 2% bovine serum albumin, 5 mM creatine, 2 mM L-carnitine, 5 mM taurine, 0.25 mM L-ascorbate, 10 μM cytosine arabinoside, 200 units/ml penicillin and 200 mg/ml streptomycin (Invitrogen). The freshly isolated adult cardiomyocytes were plated into XF-9696 Polystyrene Cell Culture Microplates and were maintained in a 37° C. incubator for two days prior to experimentation.

Respirometry assay. The OCR measurements were performed using a Seahorse Bioscience XF-96 instrument according to the protocol outlined in Beeson et al. (2010). Each experimental plate was treated with vehicle controls (DMSO<0.5%), a positive control (SRT1720, 10 μM), blank controls, and the appropriate concentration of the compound of interest. The XF-96 protocol consists of five measurements of basal OCR (1 measurement/1.5 min), injection of FCCP (0.5 μM), and three measurements of uncoupled OCR (1 measurement/1.5 min). The consumption rates were calculated from the continuous average slope of the O₂ partitioning among plastic, atmosphere, and cellular uptake (Gerencser et al., 2009). Quality control evaluations considered the basal and uncoupled rates of the vehicle control, positive control, and variances between duplicate treatment wells. Based on preliminary studies the positive threshold value was >1.15 for the mean ratio of (chemical treatment FCCP-OCR/vehicle control FCCP-OCR). This threshold is ≧1 S.D. above the historic mean for the vehicle control.

Dosing of C57BL/6 mice. Male C57BL/6 mice (NCI) were between six and eight weeks old. The mice were housed in groups of three in a temperature-controlled room, under a 12/12 h light/dark cycle. Mice were randomly assigned to either saline control or formoterol treated groups (n=3-6 mice/group). Treated mice received daily i.p. injections of formoterol for up to 72 h (100 μg/kg/day) and control mice received an equivalent volume of sterile saline.

Real time RT-PCR. Total RNA was isolated from renal cortex and heart using TRIzol reagent (Invitrogen). cDNA was synthesized from 2 μg of RNA template using SuperScript II Reverse Transcriptase kit (Invitrogen). PCR products were amplified from 5 μl of cDNA template in a 25 μl reaction containing 12.5 μl of 2× SYBR Premix (Stratagene) and 400 nM of each primer (Integrated DNA Technologies) (Supp. Table 1). The average fold induction was calculated by comparing the C_{T} (threshold cycle) of the target gene to that of tublin (the reference gene). The gene expression of the reference gene remained consistent throughout each treatment. The C_{T} of each of the technical replicates was averaged, and that average was used in the following formulas:

C_{T}(target gene)−C_{T}(β-actin)={C_{T}

ΔC_{T}(treatment)−ΔC_{T}(control)=ΔΔC_{T}

2^{(-ΔΔCT)}=fold change

Mitochondrial DNA Content. Real-time PCR was used to determine relative quantities of mtDNA content in both RPTC and mouse kidney and heart tissues. Genomic DNA was extracted using the DNeasy Blood and Tissue kit (QIAGEN, Valencia, Calif.). PCR products were amplified from 25 ng of cellular DNA in a 25 μl reaction containing 12.5 μl of 2× SYBR Premix and 400 nM of each primer.

For estimation of mtDNA in RPTC the ND6 gene was amplified. The nuclear-encoded tubulin gene was used for normalization (Supp. Table 1). For estimation of mtDNA in mice the control region (D-loop) of mouse mtDNA was amplified. The nuclear-encoded apoB gene was used for normalization (Supp. Table 1) (Fuke et al., 2011).

Computational Procedures

Molecular modeling of potential mitochondrial biogenic compounds. Modeling, simulations and visualizations were performed using MOE Version 2010.10 (Chemical Computing Group Inc.). Computational procedures were performed using a Dell XPS T3400 with an Intel Core2 Duo processor E8500@3.16 GHz with 3GB RAM using a Windows XP OS. The Sigma LOPAC and the ChemBridge DiverSet™ were searched using the Tanimoto score with MACCS structural keys (166 keys) fingerprinting (Maccs II; Molecular Design Ltd.). Using tables of the compounds represented as Simplified Molecular Input Line Entry Specification (SMILES) strings were imported into MOE as an mdb database. Molecules were rigidly aligned manually, and then subjected to MOE flexible body refinement (configuration limit 100, alpha 1, gradient test 0.01, RMSD tolerance 0.5, maximum steps 500). The ChemBridge DIVERSet™ conformers were a stochastic force field based library generated using MOE conformation import with no import filters. Total number of possible conformations per molecule was 50 and used default settings including a strain limit of 4 kcal/mol, RMSD test for structural diversity of 0.15, trans conformations were enforced, and an energy minimization gradient test of 0.01 kcal/mol. Consensus pharmacophores were calculated using a distance parameter (tolerance) maintained at the default value of 1.2 angstroms and a threshold of 100%. Pharmacophore feature projections including aromatic rings (PiN), H-bond acceptors (Don2), and H-bond acceptors (Acc2) were not included in the analysis.

Statistical Analysis. Data are presented as means±s.e.m. and were tested for normality. Data that was confirmed to have a normal distribution were subjected to one-way analysis of variance (ANOVA). The respiration data failed a normality test, therefore a Kruskal-Wallis one-way ANOVA on ranks was conducted. Multiple means were compared utilizing Dunn's post hoc test and were considered statistically different when P<0.05. RPTCs and AFCs isolated from a single animal represented an individual experiment (N=1) and were repeated until an N≧four was obtained. Rodent studies were repeated until an N≧three was obtained.

### Example 2

### β₂ Adrenoceptor Agonist Formoterol Stimulates Mitochondrial Biogenesis

β₂-Adrenergic Agonist Induces MB In Vitro

Kidney proximal tubules require aerobic metabolism to maintain high levels of ATP for transport processes. The primary cultures of RPTC utilized in this study were grown under improved culture conditions with optimized glucose-free media supplemented with 6 mM sodium lactate, and increased oxygen supply (Nowak and Schnellmann, 1995; Nowak and Schnellmann, 1996). RPTC grown under these conditions remain polarized, maintain their differentiated functions, and exhibit respiration and gluconeogenesis rates comparable to in vivo renal proximal tubule cells. The primary culture of adult feline cardiomyocytes (AFC) utilized in the study also maintain differentiated function and exhibit mitochondrial respiration similar to that observed in vivo (Mitcheson et al., 1998).

Primary cultures of RPTC and AFC have been optimized for use with the Seahorse Biosciences 96-well extracellular flux analyzer (XF-96), a multi-well plate-based assay platform that addresses the need for higher throughput cellular respirometric measurements (Ferrick et al., 2008). The XF-96 instrument uses fluorescent detectors to measure oxygen consumption rates (OCR), and can be used to identify compounds that alter mitochondrial respiration (Beeson et al., 2010). Injection of the proton ionophore carbonylcyanide p-trifluoromethoxyphenylhydrazone (FCCP) uncouples the mitochondrial membrane potential from the production of ATP increasing the OCR. It was determined that this maximum respiratory capacity could be used as a measure of MB (Beeson et al., 2010).

The FCCP-OCR increased in RPTC after a 24 hr exposure to formoterol (30 nM) relative to the vehicle control (FIG. 1A). The FCCP-uncoupled OCR was also increased in AFC after a 24 hr exposure to formoterol (10 and 30 nM) (FIG. 1B). No significant changes in FCCP-OCR were observed after exposure to isoproterenol (non-selective β-AR agonist; 10, 100, 1000 nM) or BRL 37344 (selective β₃-AR agonist; 100, 300, 1000 nM). The catecholamines dopamine, epinephrine, and norepinephrine are non-selective agonists for all three of the β-adrenoceptors (Hall et al., 1990). No significant changes in FCCP-OCR were observed in the RPTC after exposure to dopamine, epinephrine, or norepinephrine (30 and 100 nM) (FIG. 1C).

To further document that the increased OCR values were the result of MB, mitochondrial DNA (mtDNA) copy number was assessed. Relative mitochondrial DNA copy number was determined using quantitative real-time PCR and the ratio of a mitochondrial-encoded gene (NADH dehydrogenase subunit 6, ND6) to a nuclear-encoded gene (tubulin). Formoterol (30 nM) treated cells exhibited a significant 2.5-fold increase in mitochondrial copy number (FIG. 1D). These results provide strong evidence that β₂-adrenergic receptor activation induces MB in RPTC and AFC and that formoterol is a potent agonist.

β₂-Adrenergic Agonist Induces MB In Vivo

To determine whether β₂-AR agonism in vivo produces MB, male C57BL/6 mice were dosed intraperitonealy (i.p.) with 100 μg/kg formoterol every day for 1 or 3 days. Kidney and heart tissues were collected from the animals and MB was determined by assessing mitochondrial DNA copy number and the mRNA levels of multiple mitochondrial proteins.

Twenty-four hr after formoterol exposure, mRNA levels of multiple genes involved in mitochondrial regulation and function increased in kidneys. PGC-1α was induced 2.5-fold and mitochondrially-encoded ATP synthase F0 subunit 6 of transmembrane F-type ATP synthase (ATP6), ND1, and ND6 were induced 2.5-,4- and 2.5-fold, respectively (FIG. 2A). The kidneys of formoterol exposed animals also showed 2-fold induction of the nuclear-encoded mitochondrial protein NADH dehydrogenase [ubiquinone] 1 beta subcomplex subunit 8 (NDUFB8). After repeated daily exposure to 100 μg/kg formoterol for 72 hr, the kidneys exhibited 1.5-fold induction of PGC-1α, as well as a 2-fold induction of NDUFB8 and ND1 (FIG. 2B). The mtDNA copy number was increased in the kidneys of mice 72 h after repeated daily formoterol exposures (FIG. 2C).

The hearts of mice exposed to 100 μg/kg formoterol for 24 hr exhibited a marked induction of PGC-1α, ATP6, ND1, ND6, and NDUFB8 (3-, 1.5-, 2-, 2- and 2-fold, respectively) (FIG. 3A). After a repeated daily exposure to 100 μg/kg formoterol for 72 hr, the hearts of formoterol exposed animals exhibited 2-fold induction of PGC-1α, a 4-fold induction of mitochondrially-encoded ND1, and a 2-fold induction of nuclear-encoded NDUFB8 (FIG. 3B). The mtDNA copy number was increased in the hearts of mice 24 and 72 h after daily formoterol exposures (FIG. 3C). These results provide strong evidence that β₂-adrenergic receptor activation induces MB in the kidney and heart.

Formoterol-Based Pharmacophore Identifies Novel Mitochondrial Biogenics

Using formoterol as a basis for chemical similarity and pharmacophore analysis, the inventors explored chemical space that would otherwise be impractical to explore given current limitations in biological assay techniques. Pharmacophores are defined as a collection of steric and electronic features required for molecular interactions with a specific biological target structure, resulting in the activation or deactivation of its biological response (Horvath, 2011). Pharmacophore models have been developed to identify biologically active chemicals responsible for therapeutic activity (Ebalunode et al., 2011). The resulting structures can be used to predict potential pharmacology based on the assumption that compounds containing the same pharmacophore are likely to cause similar effects by targeting the same active site (Horvath, 2011). Currently, there are no known pharmacophores associated with MB. Formoterol was utilized in a ligand-based approach to identify novel compounds with a similar chemical structure and determine if they induced MB.

Formoterol was used to conduct a similarity search of the 1280 compound Sigma Library of Pharmacologically Active Compounds (LOPAC). Similarity is measured using the Tanimoto Coefficient (TC). TC is a similarity metric of one-dimensional chemical descriptors that identifies the presence of molecular elements, and facilitates rapid initial comparisons (Willett, 2006). A TC similarity search of LOPAC found 23 compounds 60% similar to formoterol. RPTC were treated with the 23 identified LOPAC compounds (5 μM) for 24 hr and examined for changes in FCCP-uncoupled OCR. This assay identified two compounds, nisoxetine and tomoxetine (also known as atomoxetine), that increase FCCP-uncoupled OCR above vehicle control (Table 1). A subsequent concentration response evaluation determined that nisoxetine and tomoxetine increased FCCP-uncoupled OCR in a concentration-dependent manner, with a minimum effective dose of 30 nM (FIG. 4A).

To confirm the specificity of formoterol, cells were pretreated for 1 hr with the β-AR antagonist propranolol (5 nM) and the β₂-AR inverse agonist ICI 115,881 (3 and 10 nM) prior to a 24 h treatment with the formoterol (30 nM). As seen in FIG. 5, both propranolol and ICI 115,881 attenuated the formoterol-induced increase in FCCP-OCR in RPTC. Although nisoxetine and tomoxetine are well characterized as norepinephrine reuptake inhibitors, there is no direct published data suggesting they activate β₂-AR. Nevertheless, because there is a report that some effects of nisoxetine are blocked by propranolol (Springer et al., 1994), the inventors determined whether propranolol and ICI 115,881 blocked the MB effects of nisoxetine and tomoxetine. Propranolol (5 nM) and ICI 115,881 (3 and 10 nM) blocked the increase in FCCP-OCR produced by nisoxteine and tomoxetine (FIG. 5).

Formoterol, nisoxetine, and tomoxetine were aligned in first two and then three dimensions based on the presence of consensus chemical features within 100% of the compounds. This alignment resulted in a pharmacophore containing six features of conserved chemical similarity (FIG. 4B). F1 is a proton acceptor, F2 and F3 are hydrophobic, F4 is a mixed feature with a cationic and proton donor, and F5 and F6 are mixed features with aromatic and hydrophobic characteristics. Distances (in Å) of the model are F1-F2: 2.53, F2-F4: 2.02, F4-F3: 1.77, F3-F5: 4.55, F5-F6: 6.56, and F6-F4: 4.76.

The 6-point pharmacophore model was used to search the ChemBridge DIVERSet™ for similar compounds. This library contains 50,080 unique drug-like compounds that represent a wide range of chemical diversity. These compounds were used to create a 1,420,467 entry conformer library. An in silico search identified 16 compounds containing all six chemical features with a RMSD<1 Å. No compounds with an RMSD>1 Å were identified, but this was expected as each feature is only ˜1 Å in diameter and six features were absolutely required leading to a minimum possible RMSD. Note that the TC search and pharmacophore search of ChemBridge have no overlapping compounds.

RPTC were exposed to the 16 identified ChemBridge compounds (5 μM) for 24 hr and examined for changes in FCCP-OCR. This assay identified three novel compounds (CB1-3) that increase FCCP-OCR above the vehicle control (Table 2). 480 random compounds were tested from the ChemBridge DIVERSet™ on the XF-96. From these 480 random compounds, thirteen were determined to be biogenic (unpublished data). The inventors extrapolate that the spontaneous hit rate of the entire data set would be approximately 2.7%. The pharmacophore performed with a hit rate of 18.8% of the compounds it chose. Using the enrichment factor equation from Pearlman and Charifson (2001), the pharmacophore selected a subset of the database 6.9 times richer in biogenic compounds than the original database.

EF=(HITS_{sampled-set}/N_{sampled-set})/(HITS_{total-database}/N_{total-database})

RPTC were exposed for 24 h to 10-3000 nM of CB1, CB2, and CB3 to determine potency in increasing FCCP-OCR. CB2 and CB3 increased FCCP-OCR in RPTC with maximal effects at 30 and 100 nM respectively, while CB1 was not effective at this concentration range (FIG. 6A). These data were incorporated into the original model and used to further refine the pharmacophore. Formoterol, nisoxetine, tomoxetine, CB2 and CB3 were aligned in both two and three dimensions and matched all six of the original features suggesting the model accurately described all of the essential features (FIG. 6B). Analysis of the final alignment revealed a unique hydrophobic feature (F7) found in nisoxetine, tomoxetine, and CB2 (FIG. 6C, Table 3).

### Example 3

### Structural Rationalization of β2-Adrenoceptor Agonists in the Regulation of Mitochondrial Biogenesis

Virtual screening and drug development for β₂-AR agonists have been explored utilizing classic SAR in conjunction with X-ray structures and known agonists (de Graff et al. 2008; Renolds et al. 2009; Procopiou et al. 2009). The use of cheminformatics including docking, fragment and pharmacophore based approaches has also been applied to study β₂-AR Abrahamian et al. 2003; Glossop et al. 2010; Tasler et al. 2010). Provided herein is a means of combining pharmacophore-based screening with a phenotypic endpoint and quantitative structure activity relationship modeling (QSAR) to identify specific β₂-AR agonists that stimulate MB. Furthermore, the resulting compounds were rationalized to the crystal structure of β₂-AR.

Materials and Methods:

Isolation of proximal tubules Female New Zealand white rabbits (1.5-2.0 kg) were purchased from Myrtle's Rabbitry (Thompson Station, Tenn., USA). Rabbit renal tubules were isolated using the iron oxide perfusion method as described in Nowak et al. 1995. The resulting proximal tubules were plated on 100-mm tissue culture-grade plastic Petri dishes constantly swirled on an orbital shaker at 80 rpm. The culture medium was a 50:50 mixture of Dulbecco's modified Eagle's essential medium and Ham's F12 (lacking glucose, phenol red, and sodium pyruvate; Gibco BRL) supplemented with and supplemented with HEPES (15 mM), glutamine (2.5 mM), pyridoxine HCl (1 μM), sodium bicarbonate (15 mM), and lactate (6 mM). Hydrocortisone (50 nM), selenium (5 ng/ml), human transferrin (5 μg/ml), bovine insulin (10 nM), and L-ascorbic acid-2-phosphate (50 μM) were added to fresh culture medium. After three days the de-differentiated cells were trypsinized and re-plated onto XF-96 Polystyrene Cell Culture Microplates at a concentration of 18,000 cells/well and maintained in a 37° C. incubator for two days prior to experimentation (Ferrick et al. 2008).

RPTC respirometric assay The OCR measurements were performed using a Seahorse Bioscience XF-96 instrument according to the protocol outlined in Beeson et al. 2010. Each experimental plate was treated with vehicle controls (DMSO<0.5%), a positive control (SRT1720, 10 μM), blank controls, and the appropriate concentration of the compound of interest. The XF96 protocol consists of five measurements of basal OCR (1 measurement/1.5 min), injection of FCCP (0.5 μM), and three measurements of uncoupled OCR (1 measurement/1.5 min). The consumption rates were calculated from the continuous average slope of the O₂ partitioning among plastic, atmosphere, and cellular uptake (Ferrick et al. 2008). Quality control evaluations considered the basal and uncoupled rates of the vehicle control, positive control (SRT1720), and variances between duplicate treatment wells. Based on preliminary studies the biogenic threshold value was <1.15 for the mean ratio of (chemical treatment FCCP-OCR/vehicle control FCCP-OCR). This threshold is ≧1 S.D. above the historic mean for the vehicle control.

Chemical Informatics Modeling: Modeling, simulations and visualizations were performed using MOE Version 2010.10 (CCG). Simulations were performed on a Dell E8500 with an Intel Core 2 Duo @3. 16 GHz using a Windows XP OS, all other computational procedures were performed using a Dell XPS M1530 with an Intel Core2 Duo processor T8300@2.40 GHz w 2GB RAM using a Windows Vista OS. Compound similarity was measured and visualized using the Tanimoto coefficient metric based on MACCS structural keys using ChemMine (Backman et al. 2011).

Pharmacophore Modeling: Structures of all screened compounds were analyzed using MOE software. Using tables of inhibitors incorporated as Simplified Molecular Input Line Entry Specification (SMILES) strings (which allow for the avoidance of ambiguity in the specification of a chemical structure via a text string) were imported into MOE as a mdb database. The pharmacophore model was generated by manual alignment of compounds and rigid refinement. Molecules were added to the pharmacophore with successive adjustment of the alignment and modification of the features. A generalized pharmacophore was created by generating features that encompassed the set of corresponding features from each individual pharmacophore. If there was overlap in a feature of type X in pharmacophores A, B, and C (features X_{A}, X_{b}, and X_{c}, respectively), a feature of type X would be created for the generalized pharmacophore and its sphere would be expanded to contain X_{A}, X_{B}, and X_{c} to give feature X_{gen}. For a set of pharmacophores developed from drugs acting upon the same receptor, there may exist features that are not shared by all pharmacophores. If a molecule significantly and adversely affected the alignment (i.e., loss of multiple features), then it was not included in the pharmacophore. Molecules were rigidly aligned manually, and then subjected to MOE flexible body refinement (configuration limit 100, alpha 1, gradient test 0.01, RMSD tolerance 0.5, maximum steps 500). Initial alignments were performed using the molecules' respective energy minima. However, as the subset of tested molecules increased, less energetically favorable conformations were incorporated to account and allow for new information. In these cases, the alignment was performed by hand and the alignment was rigidly refined to prevent thermodynamic considerations from disrupting the pharmacophore. Consensus pharmacophores were calculated using a distance parameter (tolerance) maintained at the default value of 1.2 Å and a threshold of 100%. Pharmacophore feature projections including Pi rings (PiN), H-bond acceptors (Don2), and H-bond acceptors (Acc2) were not included in the analysis.

Pharmacophore virtual database searching: The ChemBridge DIVERSet was searched using pharmacophore models. Using tables of the compounds represented as Simplified Molecular Input Line Entry Specification (SMILES), strings were imported into MOE as a mdb database. The 5 point pharmacophore model was used to search the Chembridge DIVERSet. The DIVERSet database contained 50,080 unique compounds representing diverse drugs designed for primary drug discovery. The chemical graphs of the DIVERSet were used to create a 2,500,000 compound conformer library, using MOE conformation import and allowing a maximum of 250 conformers per compound and a strain limit of 4 kcal. Using the five point CB pharmacophore model conformers were matched to the model which required all features to be present. In order to match all five features an RMDS threshold of <1\acute{Å} was a functional upper boundary due to the radius (max=1.2 \acute{Å}, avg=0.72 \acute{Å}) of each feature.

2D-QSAR modeling and screening: A 2D-QSAR models were built that showed significant predictive ability. QSAR modeling correlated activity as a binary function to the calculated 2D descriptors. Due to the large divergence in chemical space the original compound list was pruned to 19 compounds, representing 12 active and 7 inactives. In total 186 2D descriptors were calculated. These were trimmed though integrative modeling and removal of low predictive descriptors to a final set of 13 descriptors. The final cross-validated model was saved as a .fit file and used to search 2D chemical mdb libraries.

in silico receptor interaction studies: The interaction of ligands from the compound library were analyzed for molecular interactions. The consensus pharmacophore model was used as a placement strategy. The consensus pharmacophore was aligned manually within the β₂-AR Xray structure (pdb 3NYA (Wacker et al. 2010)). The alignment was guided by overlaying the model on top of alprenolol. After pharmacophore placement, alprenolol was removed from the system and modeled ligands placed according to the conformer from the pharmacophore model. The ligand-receptor dimer system was then energy minimized using OPLA-AA. Some modest manual adjustment was required to prevent van der Walls coincidence that would negatively affect the energy minimization. The minimized system was then analyzed by visualization of key interactions and ligand interaction maps.

### Results:

### Screening of β₂ Agonists and Similar Compounds in RPTC Identifies MB Chemicals.

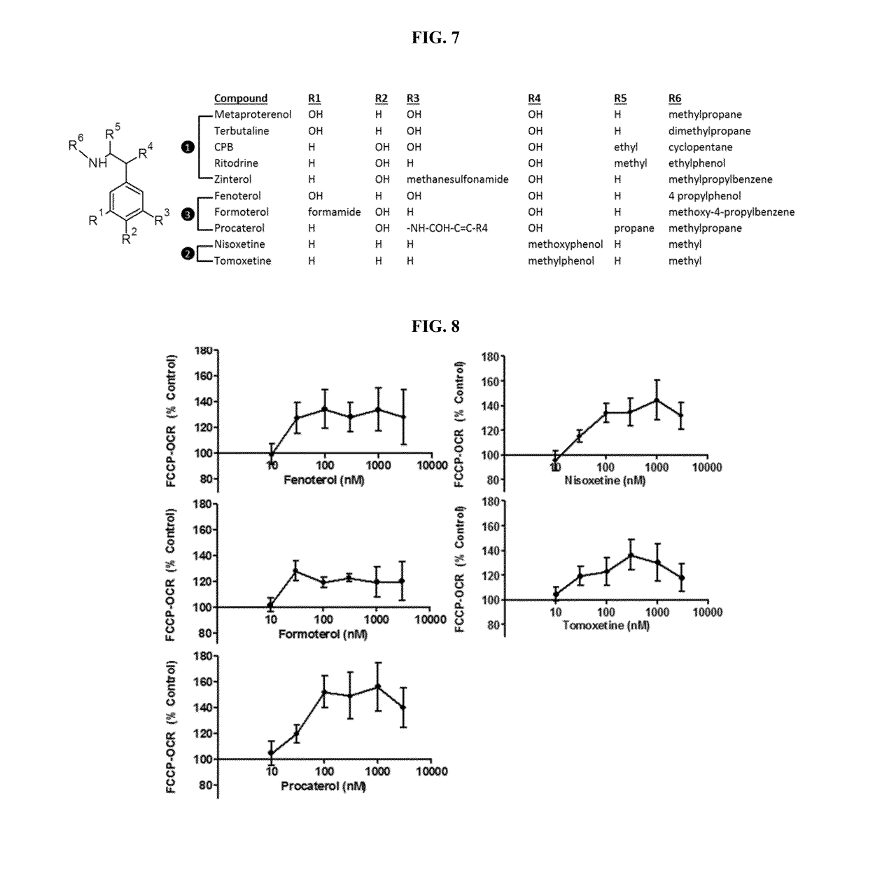

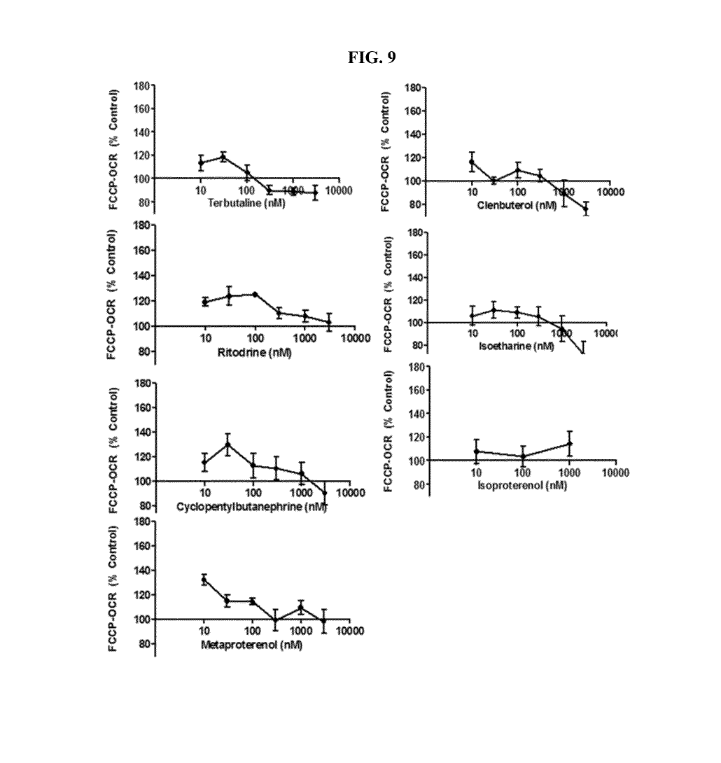

To expand understanding of β₂-agonist-stimulated MB, the inventors tested β₂-AR agonists and similar ligands. Chemotype evaluation revealed that ten compounds contain a phenethylamine core common to many biogenic amine ligands that are β-AR specific (FIG. 7). RPTC were exposed to increasing concentrations (10-3,000 nM) of these compounds and 24 h later FCCP-OCR was determined (Beeson et al. 2010). Chemicals were classified as MB based on a mean ratio of: (chemical-treated FCCP-OCR)/(vehicle control FCCP-OCR)>1.15. This value is ≧1 standard deviation (SD) from the mean ratio for the vehicle control. From this analysis nine compounds were identified that increased FCCP-uncoupled OCR compared to the vehicle control at 24 hr (FIG. 8 and FIG. 9).

There were three types of concentration-response phenotypes. Five of the twelve compounds tested were very potent, had Michaelis-Menten type concentration-response curves and included fenoterol, formoterol, procaterol, nisoxetine and tomoxetine (FIG. 8). A second set of four ligands (terbutaline, ritodrine, cyclopentylbutanepharine, and metaproteranol) were very potent at low concentrations but were not MB as concentrations increased (FIG. 9A). A final set of three ligands (clenbuterol, isoetharine, and isoproteranol) had no effect on MB, although they are within the same chemotype as formoterol (FIG. 9B). These data indicate that subtle manipulations of ligand structure can alter potency and efficacy of MB mediated by the β₂-AR. Salbutamol and salmeterol were also examined and were found to be inactive at stimulating MB. The β₃ agonist BRL 37344 was also previously reported to be inactive (Wills et al. 2012).

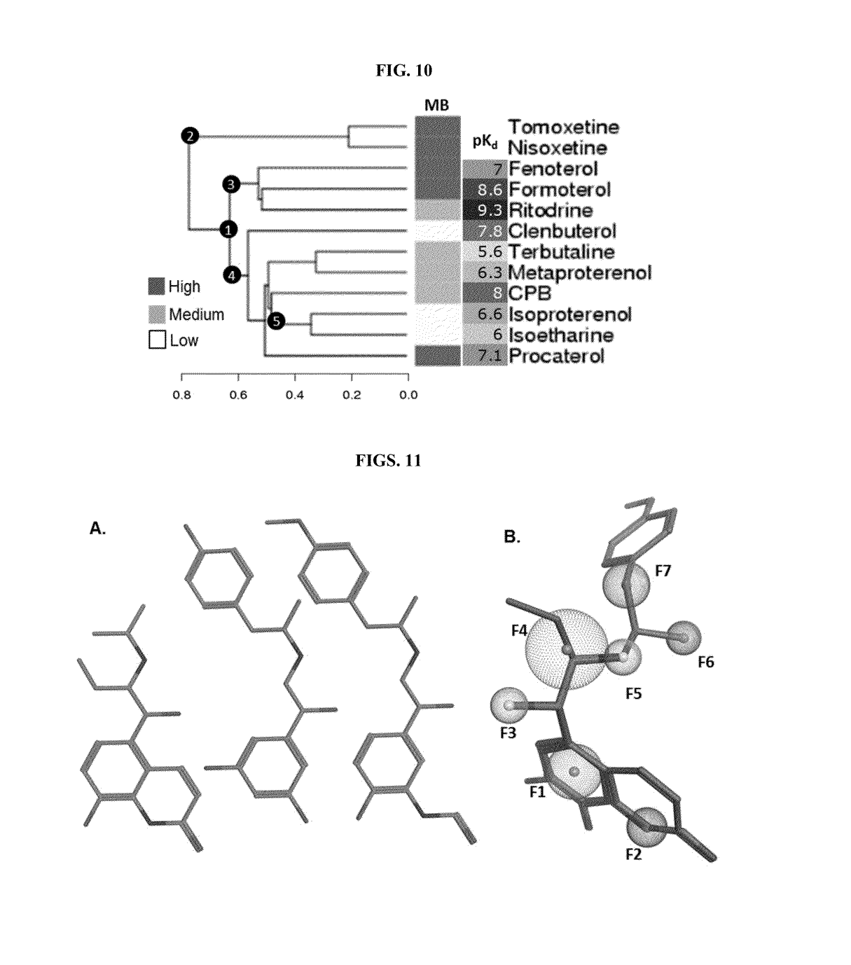

Due to the discrepancy in the ability of high affinity and selective β₂-AR ligands to stimulate MB, the inventors interrogated the ligand structure using chemical similarity clustering (FIG. 10). Clustering was performed using Chemmine Tc based fingerprinting with single linkage hierarchical clustering (Backman et al. 2011). Two major clusters were identified that separated all the classical β₂-AR agonists (cluster 1) from nisoxetine and tomoxetine (cluster 2). It should be noted that while nisoxetine and tomoxetine induce MB, they are not known to bind nor activate the β₂-AR. Within the classical β₂-AR cluster there were two sub-clusters that separated fenoterol, formoterol and ritodrine (cluster 3) from the rest of the classical agonists (cluster 4). Within cluster 4, isoproterenol, isoetharine, and clenbuterol did not exhibit any MB activity. This cluster would include other partially selective and inactive β₂-AR agonists like epinephrine and norepinephrine which were previously shown to not stimulate MB (Wills et al. 2012). It is important to note that MB potential does not correlate with any particular chemical cluster using this approach nor reported pK_{d} for the β₂-AR (FIG. 10). These data reveal that there are subtle but discrete chemical changes required for promoting MB.

Cluster 1 was further explored using pharmacophore modeling because their chemical similarity did not explain their biological effect. Procaterol, formoterol and fenoterol were aligned by hand based on steric considerations. The alignment was then refined using MOE's flexible alignment tool. The final pharmacophore model generated had 100% consensus features with a maximum feature radius of 1.2 Å (FIG. 11).

Features F1, F3, F4, and F5 represent the phenethylamine core found in many sympathomimetic compounds as well as the endogenous sympathetic agonists. Features F6 and F7 are an expansion in the hydrophobic space occupied by this core scaffold. Given that neither epinephrine nor norepinephrine elicit the MB nor contain these features, these features occupy necessary space for MB. Features F1, F3, F4, and F5 represent the phenylethanamine core found in many sympathomimetic compounds as well as the endogenous sympathetic agonists. Features F6 and F7 are an expansion in the hydrophobic space occupied by this core scaffold. Given that neither epinephrine nor norepinephrine elicit the MB nor contain these features, these features occupy necessary space for MB. However, because non-biogenic compounds (e.g., isoproterenol) contain these features, they are not sufficient for mitochondrial biogenesis. Feature F2, although present in all the fully biogenic β₂ agonists, is present in non-biogenic agonists and is not present in clusters 2 or 3; therefore, it is neither necessary nor sufficient in itself for mitochondrial biogenesis.

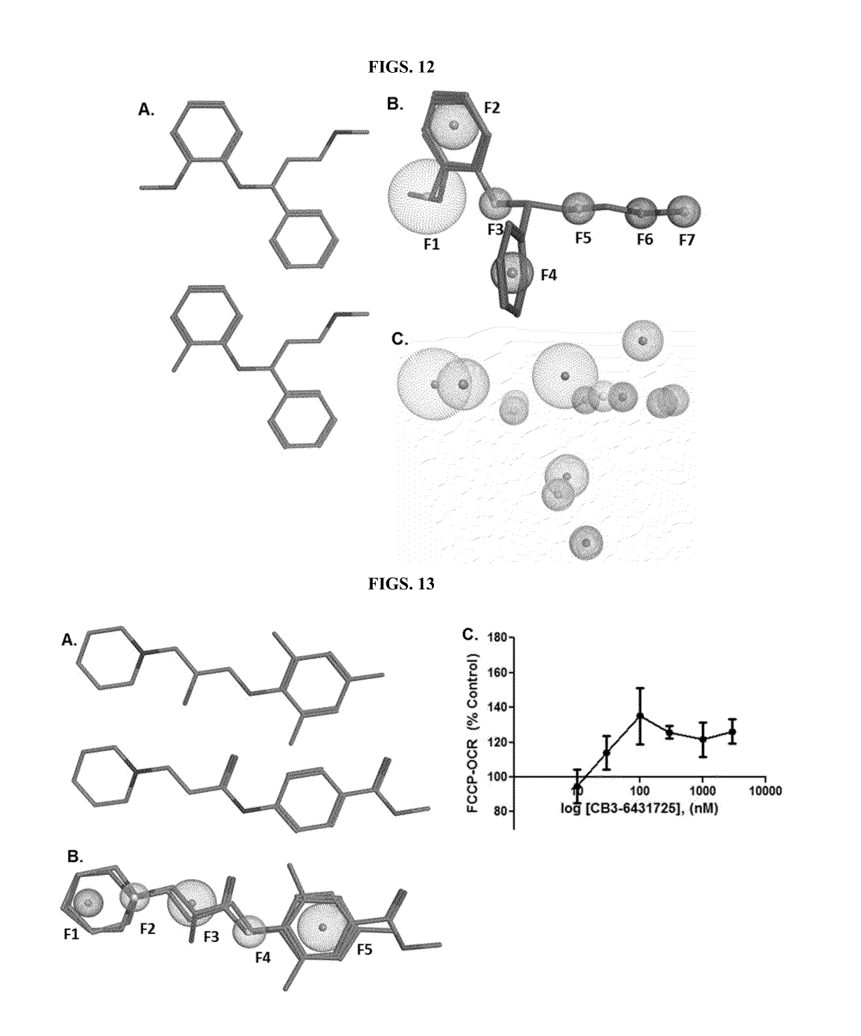

Cluster 2 contained nisoxetine and tomoxetine, which are characterized as norepinephrine reuptake inhibitors (NRIs) and represent non-classical but chemically similar compounds to β₂-AR agonists that stimulate MB. Nisoxetine and tomoxetine were aligned in first two and then three dimensions. A high quality model with seven features and tight spheres was generated (FIG. 12). Features F4, F5, F6, and F7 in this pharmacophore represent the phenethylamine core. However, unlike the cluster 1 pharmacophore, there were no further hydrophobic substituents on the carbon alpha to the nitrogen hydrogen bond donor group. This indicates that Features F1 and F2, absent from the cluster 1 pharmacophore, are sufficient to stimulate MB when coupled with the phenethylamine core.

Features F3, F4, F5, F6, and F7 in this pharmacophore represent the phenethylamine core. However, unlike the Cluster 1 pharmacophore, there were no further hydrophobic substituents on the carbon alpha to the nitrogen hydrogen bond donor group. This indicates that features F1 and F2 (hydrophobic acceptor and aromatic, respectively), absent from the cluster 1 pharmacophore, are sufficient to stimulate MB when coupled with the phenethylamine core. However, due to its absence in cluster 3, F2 alone may not be sufficient to generate a biogenic phenotype.

The potential overlap of cluster 1 and cluster 2 was then determined (FIG. 12C). The primary positioning parameter was the overlap of corresponding features with hydrogen-bonding interactions. Five features had both chemical and spatial overlap. Two of the conserved features represent the phenethylamine core and three more features conserved between cluster 1 and cluster 2 compounds was found. This left two discrete features with one derived from each model.

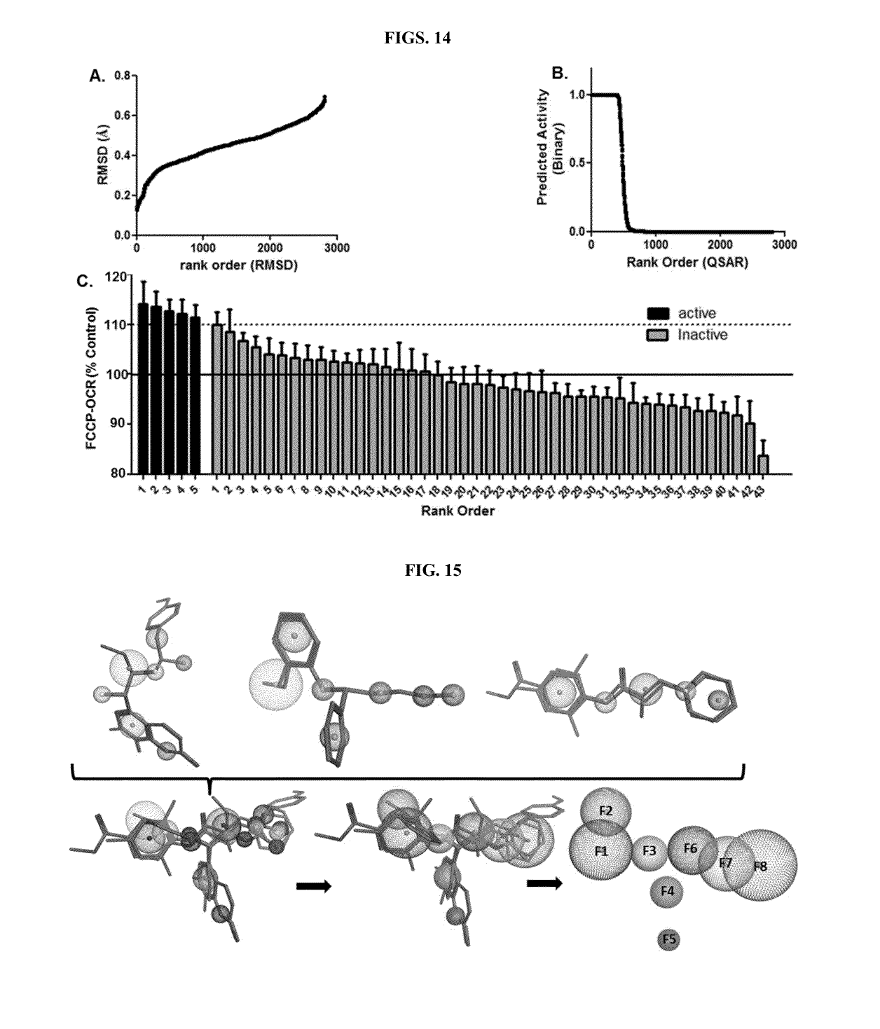

Because the above models and clusters only provide information relevant to the phenethylamine core, the inventors expanded on the discovery of the MB compounds CB2 and CB3 (FIG. 13), which do not have a phenethylamine core (Wills et al. 2012). CB3 promoted full MB in a full concentration-response (FIG. 13B). A discrete model of both CB2 and CB3 was developed and contained five unique features (FIG. 13B). The five-point pharmacophore model was used to virtually search the Chembridge DIVERSet (FIG. 14A). The CB pharmacophore model aligned with 56207 conformers representing 2810 discrete compounds. This represents 5.6% of the total 50,080 compound database, and 0.2% of the 2.5 million possible conformers.

To further limit the chemical space being tested, the results of the CB pharmacophore search were refined using a discrete 2D-QSAR model. A preliminary 2D-QSAR model was built from all of the tested compounds using a pharmacophore-based on the cluster 3 pharmacophore. Binary QSAR modeling was used to account for the inability to determine IC₅₀ measurements or perform linear QSAR with OCR data. The cross-validated predicative statistics for the QSAR model were 86% for actives and 100% for inactive with a p-value <0.001. If a random hit rate of 3% is assumed, this process gave an enrichment of 3.5× over random. When used together the pharmacophore model and 2D-QSAR yielded 1196 from the 2810 discrete compounds. To further limit the test set the inventors further applied an RMSD filter of <0.3 Å to the pharmacophore results to further refined and narrowed the results to 48 chemicals.

The 48 compounds predicted by pharmacophore and QSAR modeling were tested for their ability to induce MB at 30 nM in RPTC. Five of the 48 compounds (10%) produced MB, resulted in the expansion of the original chemogram, shows that the newly characterized compounds diverge from classical β₂-AR chemical space, and demonstrates how directed chemical modeling can explore new chemical clusters with similar biological properties. A workflow for modeling for combined QSAR and pharmacophore modeling for virtual screening was used.

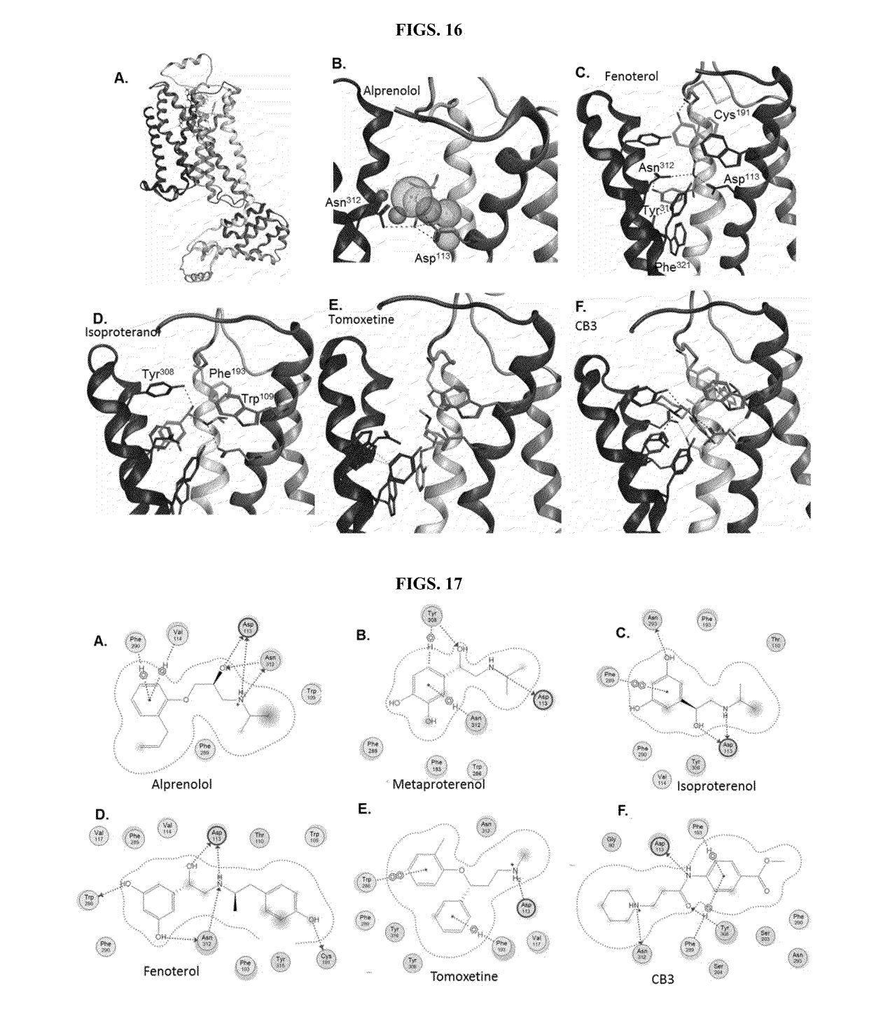

Finally, the inventors investigated whether there were conserved features (FIG. 15) between the combined cluster 1&2 pharmacophore (FIG. 12C) and cluster 3 pharmacophore (FIG. 13B). As in the consensus model between cluster 1 and 2 the inventors rigidly overlaid the three models using the core hydrophobic features as a key and maximized matching of chemical features. The overall consensus model had eight features and five areas of consensus overlap between the three models. The three unique features were each derived from each discrete model. These data suggest all of the MB compounds share a portion of the binding mode, but each cluster utilizes at least one discrete interaction providing a mechanism for the bias.

In the consensus pharmacophore, F3, F4, F6, and F7 represent the phenethylamine core. F5 is a donor feature in the meta position to the ethanamine portion of the core and is unique to the Cluster 1 pharmacophore. Because it is not found in the other clusters and is also present in non-biogenic compounds, it is neither necessary nor sufficient for mitochondrial biogenesis. However, it may play a role in stabilizing receptor interactions of individual compounds to generate a conformational change that induces biogenesis. F1 and F2 represent the aromatic feature and hydrophobic acceptor features from cluster 2, respectively. F1 is present in clusters 2 and 3 and is sufficient to cause biogenesis when added to the core. F2 is only present in cluster 2 and is therefore neither necessary nor sufficient. Likewise, the donor feature F5 is present only in cluster 1 and is also neither necessary nor sufficient. The hydrophobic feature F8 is present in all clusters and is therefore a necessary addition to the phenethylamine core in stimulating mitochondrial biogenesis.

The MB ligands were analyzed for predicted interactions with the β₂-AR X-ray structure (Ghanouni et al. 2001; Suryanarayana et al. 1992; Rasmussen et al. 2011; Rosenbaum et al. 2007; Wacker et al. 2010). Using the structure of the β₂-AR bound to the antagonist alprenolol (pdb: 3NYA (Wacker et al. 2010)), the consensus pharmacophore was utilized as a placement strategy to identify potential high-likelihood interactions (Audet et al. 2008). Alprenolol was chosen over other available structures due to the number of chemical similarities of the antagonist to the pharmacophore models. The inventors observed overlap between the donor feature from the cluster 1 pharmacophore and Phe²⁸⁹ or Phe²⁹⁰ (depending on the ligand placement) within the ligand binding pocket without overlapping with any receptor amino acids indicating the pocket has room for the multiple conformations predicted by modeling (FIGS. 16A-F). The specific conformer of each biogenic ligand was placed within the ligand binding site of the receptor. The final agonist-receptor complex was energy minimized and analyzed for potential interactions.

Common residue interactions were found across the full biogenic agonists (FIGS. 17A-F) (Audet et al. 2008; Vilardaga 2006). Across the classic β agonists as well as nisoxetine and tomoxetine, all of which contain secondary amines, Asp¹¹³ was found to interact with the ethanamine core. However, in the novel Cluster 3, Asp¹¹³ only interacted with the secondary amide rather than the tertiary amines (both of which were protonated, giving a coulombic electrostatic interaction in addition to the normal hydrogen bonding interactions). This indicates that Asp¹¹³ is unlikely to interact with tertiary amines. However, Asn³¹² interacted with various substituents across clusters and phenotypes, including the tertiary amines of the novel cluster. Furthermore, across all conformations, Asp¹¹³ formed a hydrogen-bonding interaction with Tyr³¹⁶, creating an interaction across helices.

In the first cluster of classical β₂ agonists (cluster 1), formoterol had extensive hydrogen bonding interactions, with its alkyl hydroxyl group interacting with both Asp¹¹³ and Asn³¹². The p-OH group interacted with the backbone of residues Trp²⁸⁶ and Phe²⁸⁹. Hydrophobic interactions occurred between Tyr³⁰⁸ and the formamide hydrogen and between Phe¹⁹³ and the methoxyphenyl ring. These interactions, and especially that of Asp¹¹³, differ greatly from the other ligands due to the extra space occupied by the formamide chain. This is further demonstrated by the interactions between the receptor and fenoterol. The resorcinol hydroxyl groups on fenoterol interact with the receptor (Asn³¹² and the backbone of Trp²⁸⁶). Asn³¹² also interacts with the amine on the alkyl chain, as does Asp^{113.} Asp¹¹³ in turn interacts with the hydroxyl of the ethanolamine core. The p-OH distal to the phenethylamine core interacts with Cys¹⁹¹. Finally, the phenyl ring of the phenethylamine core is in proximity to Phe²⁸⁹, suggesting the potential for a it-stacking interaction. Despite structural similarities to formoterol, the small differences result in distinct conformational changes of both the receptor and the ligand. A similar phenomenon arises with procaterol. Although it lies in the same chemical cluster as fenoterol and formoterol, its interactions include a it-stacking interaction between Phe²⁸⁹ and the quinoline system, a hydrophobic interaction between Tyr²⁰⁸ and the quinoline ring, a hydrophobic interaction between Phe¹⁹³ and the N-isopropyl system, and a hydrogen bond between the amine and Asp¹¹³. This π-stacking interaction is conserved among MB compounds and is facilitated in procaterol due to the size of its aromatic system.

The second cluster containing nisoxetine and tomoxetine conserved the Asp¹¹³ interactions with their secondary amines. Tomoxetine also had a hydrophilic interaction with Ser²⁰³ and the methoxy group and a hydrophobic interaction between Trp¹⁰⁹ and the N-methyl group, while the phenyl rings of tomoxetine had a hydrophobic interaction with Phe¹⁹³ and a π-stacking interaction with Trp²⁸⁶. Despite the remarkable similarity of these compounds, the methoxy group on nisoxetine allows for hydrophilic interactions that place it in a certain area of the binding pocket. This allows for the difference in interactions between two compounds that are so similar.

The NRI's represent a vast chemical space, but nisoxetine and tomoxetine represent a distinct and limited class of NRI's (Civitarese et al. 2008). These drugs have less use due to the recognition of the promiscuity in mechanism, although they have only recently been proposed to potentially stimulate the β₂-AR (Wills et al. 2012). Other NRIs lack the features described here found necessary for supporting β₂-AR mediated MB. One example is desipramine, which has a tail secondary amine; however it is missing all the alkoxy and hydroxy moieties including the classical ethanolamine and more specifically the acceptor feature (F3) from the models (FIG. 12). This data also indicate that common features found in many other NRIs including tricyclic systems (desipramine, mazindol, tandamine, ciclazindol) or substituted indenes (amedalin, daledalin, talopram, and talsupram) would not be supportive of MB, and are structurally more dissimilar form the β₂ type compounds.

Both compounds of novel cluster 3 have interactions between Asn³¹² and the tertiary amine. CB2 has interactions between two of its CH₂ components with Phe¹⁹³. CB3, however, has a hydrophobic interaction between residues Phe¹⁹³ and Phe²⁸⁹ and the phenyl system. It also has a hydrophilic interaction between Tyr³⁰⁸ and its amide carbonyl group. Once again, despite the chemical similarity based on Tc, these compounds differ greatly in their possible 3D interactions with the receptor.

The partial biogenic compounds were ambiguous, requiring multiple alignments to achieve a conformation sampling the canonical residues (i.e., Asp¹¹³ and Asn³¹²) (Suryanarayana et al. 1992; Rasmussen et al. 2011; Wacker et al. 2010; Audet et al. 2008; Vilardaga 2006; Suryanarayana et al. 1993). These data reveal that MB compounds can transiently and rapidly alter conformations, resulting in their partial biogenic phenotype. In the case of CPB, the cyclopentyl group makes the ligand too large to rapidly alter its conformation preventing the transient interactions with the residues necessary for MB. One of the potential conformations had a it-stacking interaction between Phe²⁸⁹ and the CPB phenyl group. This is similar to the full agonist procaterol, which also potentially interacts with Phe²⁹⁰. The hydrophobic interactions between the cyclopentyl group and the binding pocket may aid in dynamic receptor conformation sampling as it tries to find an appropriate minimal energy conformation. This is in contrast to isoetharine, which has an isopropyl group rather than a cyclopentyl group. The lack of volume distal to the phenyl group prevents the ligand from being pressed deeper into the binding pocket, thereby preventing the necessary biogenic interactions with the receptor.

The metaproterenol analysis underwent multiple conformations, all sampling at least one of the canonical residues. This indicates that the partial agonist activity of metaproterenol results from being too small to adequately remain in the binding pocket. Its small size therefore allows for it to easily sample the necessary residues for biogenesis, but it does not allow it to stably and simultaneously sample them. Compared to isoproterenol, which has a catechol ring rather than a resorcinol ring, metaproterenol has twice as many conformations available to it that sample the same residues. Since it has two m-OH groups rather than an m-OH group and a p-OH group, it can more easily sample the it-stacking interactions with F²⁸⁹. This structural difference helps to explain the differences in receptor-ligand interactions (and therefore the phenotypic differences) between isoproterenol and metaproterenol.

In this study a recently developed phenotypic respirometric assay was utilized to screen for MB agents in primary RPTC. A phenotypic assay has the major advantage of linking specific ligand-mediated receptor activation (i.e. β₂-AR) to a distinct cellular function. Although FCCP-OCR is a one dimensional parameter it is reflective of a complex process and ideal for elucidating signaling bias. Furthermore, using primary cultures of RPTC have more scientific and clinical relevance than similar assays in cell lines, due to the reliance of RPTC on aerobic respiration.

17 MB agents were identified, three major chemical clusters, three cluster specific pharmacophore models, a QSAR model and a consensus pharmacophore model. Three distinct OCR phenotypes were observed, including full biogenic, partial biogenic, and non-biogenic activities amongst the agonists and similar compounds. The inventors rationalized the chemical structures of β₂-AR ligands with their ability to regulate MB, and determined that the reported K_{d} of these compounds are not in agreement with the ability to produce MB. The inventors rationally expanded the chemical space available for MB to include defined NRIs and uncharacterized compounds. The consensus pharmacophore model derived from three discrete pharmacophore models allows for the precise spatial orientation of the alternate chemical moieties that potentially extend the core model towards a biased model. Furthermore, the model reveals there are at least three discrete interactions that potentially create the biased effect.

There is still insufficient detail on the structural rationale behind the design of biased agonists. Molecular docking has been used in the discovery of β₂ agonists and antagonists using the GPCR ligand binding site, but the atomic basis is still an area of active investigation (Baloyannis et al. 2004; Ktritch et al. 2009). While the inventors initial thought was that the MB potential of the compounds would correlate with their reported affinity, there appears to be no relationship between K_{d} and MB potential (FIG. 10). This still leaves many properties including duration of signaling and receptor occupancy that could have profound effects on the observed biology. Metaproterenol has the greatest OCR at the lowest dose but is only intermediate in terms of affinity for the receptor. Comparing metaproterenol to the full biogenic compounds and assuming that receptor density did not change indicates full receptor occupancy might not be necessary for full biogenic response. It is interesting to note how dissimilar the absolute chemical structures are between compounds like formoterol, nisoxetine and CB3 are, but their 3D chemical features are nonetheless in tight correlation in chemical space.

The inventors elucidated a simple binary 2D-QSAR model consisting of 13 descriptors. Binary QSAR leads to a marginal decrease in absolute predictive power, while still allowing an orthogonal methodology compared to pharmacophore modeling. The fact that the developed model was refined to only 13 descriptors indicated that there are limited and specific sets of requirements. Binary QSAR also identified the conserved electronic chemical features consistent with bioactivity. Analysis of the top descriptors used in the 2D-QSAR model provides insight into the chemical properties that lead to biogenic capability (Peterson et al. 2009). The top descriptor was the number of oxygen atoms indicating dependence on a proton acceptor. The number of aliphatic hydroxyl groups (avg=2.18 per molecule) and nitrogen atoms (avg=1.36) was also necessary indicating a need for protein donors (avg=1.5). The HBD and HBA descriptors, which represent counts of hydrogen bond donors and hydrogen bond acceptors, came up in four forms indicating the necessity for reversible non-covalent interactions to support MB. The remaining descriptors indicated a limit to the number and types of intramolecular bonds: # bonds (avg=41.46), # heavy bonds (avg=20.36), # atoms (avg=40.72), # rotatable bonds (avg=5.72). All these data indicate that the biased MB potential of the ligands is related to the non-covalent and flexible interaction of the drug with its receptor.

The partial and non-biogenic compounds have classical architecture incorporating both ethanolamine and either catechol or resorcinol moieties while none of the full biogenics have a catechol. From a chemistry perspective isoetharine and procaterol are bioisosteres of one another, with procaterol having a quinoline system rather than a catechol. However, procaterol is strongly biogenic, while isoetharine is not, indicating that the space occupied by the quinoline ring is also important for the MB phenotype. Furthermore, CPB exhibits a partial biogenic response while differing from isoetharine by only two carbons. This indicates that, in addition to the stronger hydrogen bond interactions with the receptor, weaker hydrophobic interactions play an important role in differentiating phenotypic responses in the β₂-AR. There were more hydrophobic interactions in the full biogenics in these simulations compared to the other two classes of compounds. It is interesting to note that no simulated interactions with Tyr³¹⁶, a member of the canonical polar triad for agonist binding, was observed. It is possible this triad is responsible for the activity of full endogenous agonists and during ligand simulations the inventors repeatedly found that Tyr³¹⁶ formed a stabilizing bond with Asp¹¹³, which has been previously been associated with agonist activity (Katritch et al. 2009). However, the prevalence of the phenethylamine core indicates that hydrogen bonding interactions is still critical in ligand binding in the receptor pocket to allow slower hydrophobic interactions to occur.

Understanding of GPCR pluridimensional signaling has led to the conception of manipulating pharmaceutical design for functional selectivity (Galandrin et al. 2006; Kenakin et al. 2010; Luttrell et al. 2011; Stallaert et al. 2012). The mechanism of MB and indeed of signaling bias is not yet known and not generalizable to a particular chemical class or chemotype. It is possible the three MB phenotypes represent three distinct states of the receptor that the ligands are able to bias. The ability of different orthosteric ligands to induce a spectrum of receptor conformations allows for multiple signaling states of GPCRs. In such a model, the ligand receptor complex, not solely the receptor, specifies the activation state and is therefore the minimal pharmacological interpretation unit. In the case of the MB β₂-AR agonists, the non-overlapping features were inferred to allow for biased agonism by stabilizing different conformations of the receptor (Reynolds et al. 2009; Audet et al. 2008; Sauliere et al. 2012; Seifert et al. 2001). By allowing for the stabilization of multiple conformations with a single compound, it should be possible to sample multiple pathways with a given binding event and thereby potentiate MB. Furthermore, by utilizing this same process of compound development for other desired effects, it should be possible to attenuate negative effects in addition to enhancing desired pathways.

The overall therapeutic paradigm for usage of MB agents may be performed using short term and low dose administration in order to boost mitochondrial function resulting in the stimulation of cellular/organ repair and regeneration. This preliminary structural rationale for MB compounds is not specific to a chemical class, but a binding mode. Not unexpectedly, it is the ligand-receptor complex that dictates signaling. The pharmacophore models can be utilized to develop novel compounds with biogenic potential that minimize the deleterious effects sometimes associated with activation of the β₂-AR. This would allow for the use of compounds over a longer time course to treat chronic mitochondrial problems.

### Example 4

### Tomoxetine Promotes Healing of Muscle Wasting

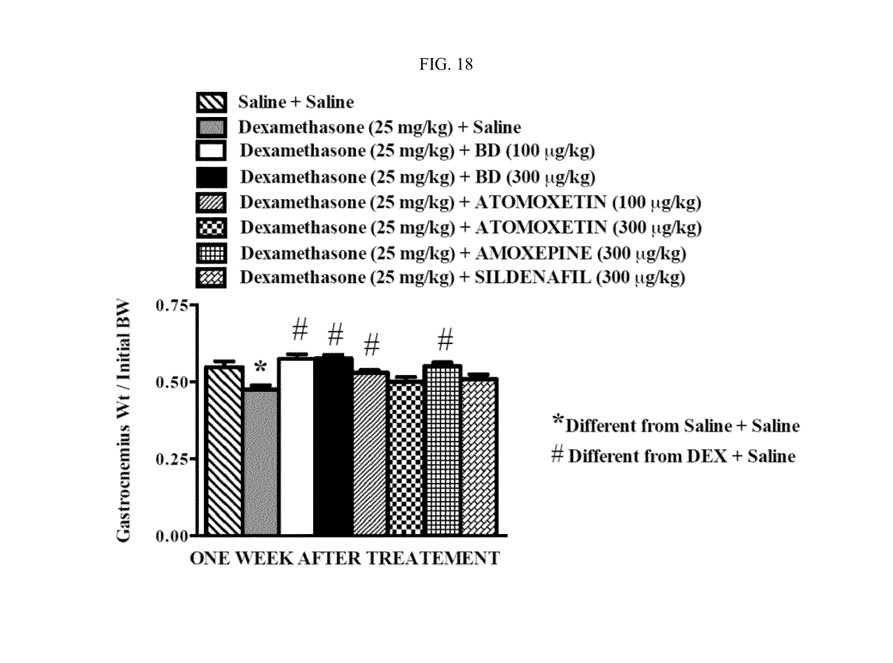

Mice were co-injected with either saline or dexamethasone (25 mg/kg, i.p.) followed by a second injection with either saline or formoterol (BD 100/300/ ug/Kg, i.p.) or tomoxetine (“atomoxetine” 100 ug/kg, i.p.) for 7 days. On the 8^{th} day, gastrocnemius muscle was isolated from both the hind limbs and weighed. The average weights were normalized to initial body weights. DEX caused a 15% decrease in gastrocnemius wt. when compared to saline controls. Both formoterol and atomoxetine administration restored atrophied muscle back to normal. Results are shown in FIG. 18.

### Example 5

### Formoterol Promotes Mitochondrial Biogenesis And Restores Renal Function Post Ischemic-AKI

Materials and Methods

I/R model of AKI. Eight-week-old male C57BL6 mice weighing 25-30 g were subjected to bilateral renal pedicle ligation. Briefly, renal artery and vein were isolated and blood flow was occluded with a vascular clamp for 20 min, and mice were euthanized at 24, 72, or 144 h after procedure, at which time kidneys were harvested for molecular analysis. All procedures involving animals were performed with approval from the IACUC in accordance with the NIH Guide for the Care and Use of Laboratory Animals.

Assessing renal function. For mice, blood was collected by retro-orbital eye bleed 24 h and 144 h. Creatinine levels were measured using a Quantichrom Creatinine Assay Kit (BioAssay Systems, Hayward, Calif.) according to the manufacturer's protocol.

Immunoblot analysis. Renal cortical tissue was lysed in RIPA buffer containing cocktail protease and phosphatase inhibitors. Total protein content was measured by the BCA assay. Fifty micrograms of total protein were loaded into SDS-PAGE gels and immunoblots were performed as previously described (26). Antibodies used for immunoblot studies were obtained from the following vendors: caspase 3 (Enzo Life Sciences), GAPDH (Fitzgerald Antibodies), COX I and NDUFB8 (MitoSciences), KIM-1 (R&D Systems), and PGC-1α (Calbiochem).

mRNA analysis. Total RNA was isolated from renal cortical tissue with TRIzol (Invitrogen) according to the manufacturer's protocol and cDNA was synthesized using RevertAid First Strand cDNA Synthesis Kit (Fermentas). PCR reactions were performed using 3:1 diluted cDNA product. Primers sequences for NDUFB8 and COX I were used.

Formoterol Restores Kidney Function After I/R Injury

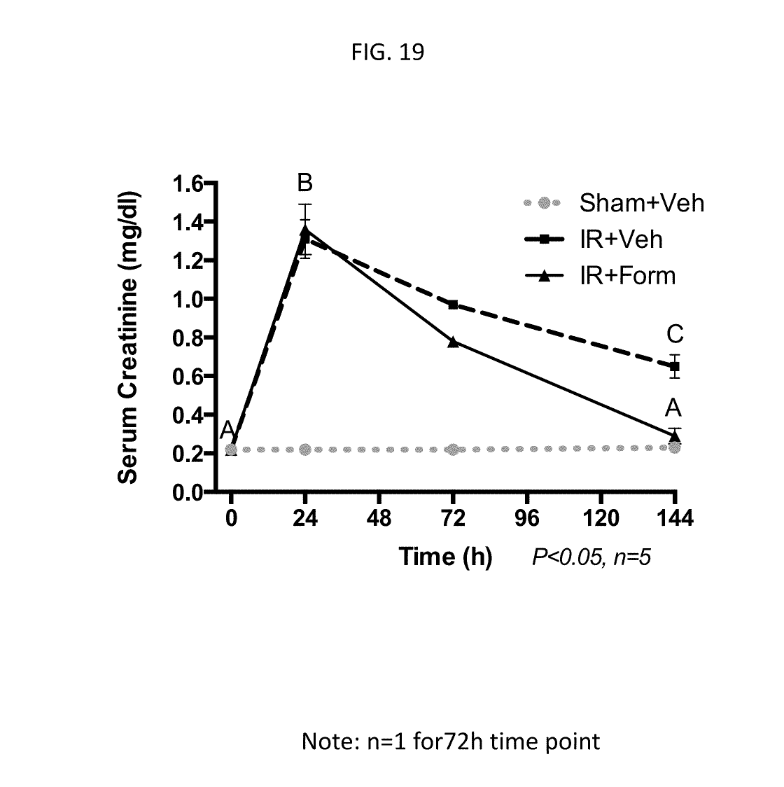

Initially, to identify the most clinically relevant and translatable time of dose for formoterol to restore kidney function the inventors defined the time of maximal injury, as determined by serum creatinine (SCr), after I/R induced AKI. Maximal injury was observed at 24 h, SCr was approximately six fold higher than that of sham animals. It was at this time that administration of formoterol was initiated. After five doses the inventors observed complete recovery in SCr, 0.29 mg/dL. However, in the absence of formoterol SCr remained persistently elevated by approximately three fold higher compared to the control group at 144 h after I/R injury (FIG. 19).

Formoterol Rescues Proximal Tubules from Damage After I/R Injury

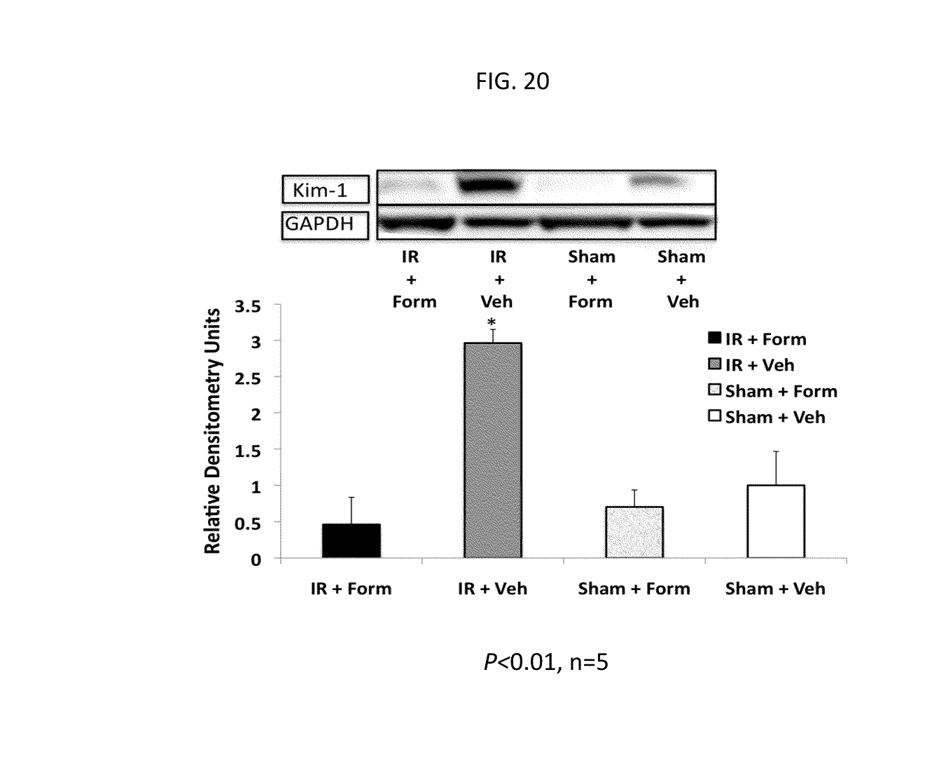

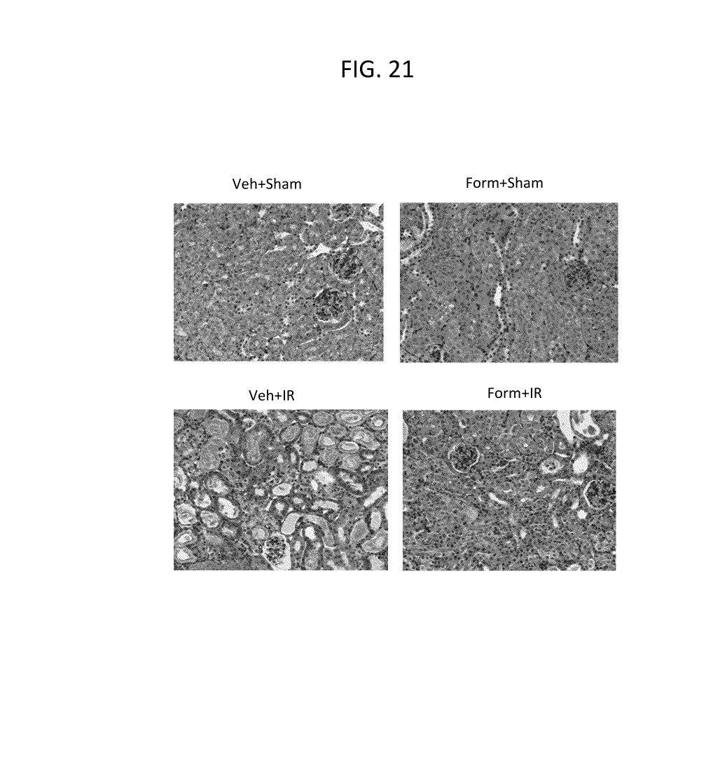

Kidney injury molecule-1 (Kim-1) is a marker specific to tubular injury and is ultimately undetectable in healthy kidneys (65,66). Therefore, the inventors evaluated if administration of formoterol after I/R injury was capable of decreasing the KIM-1 protein expression. It was discovered that administration of formoterol after I/R injury restored expression of KIM-1 at 144 h post injury to the level of sham vehicle. In contrast, the I/R vehicle group showed a three-fold increase, over vehicle, in KIM-1 protein expression out to 144 h (FIG. 20). Effect of formoterol on renal cortical morphology after I/R injury are shown in FIG. 21.

Effect of Formoterol on Expression of PGC-1α and ETC Components after I/R Injury

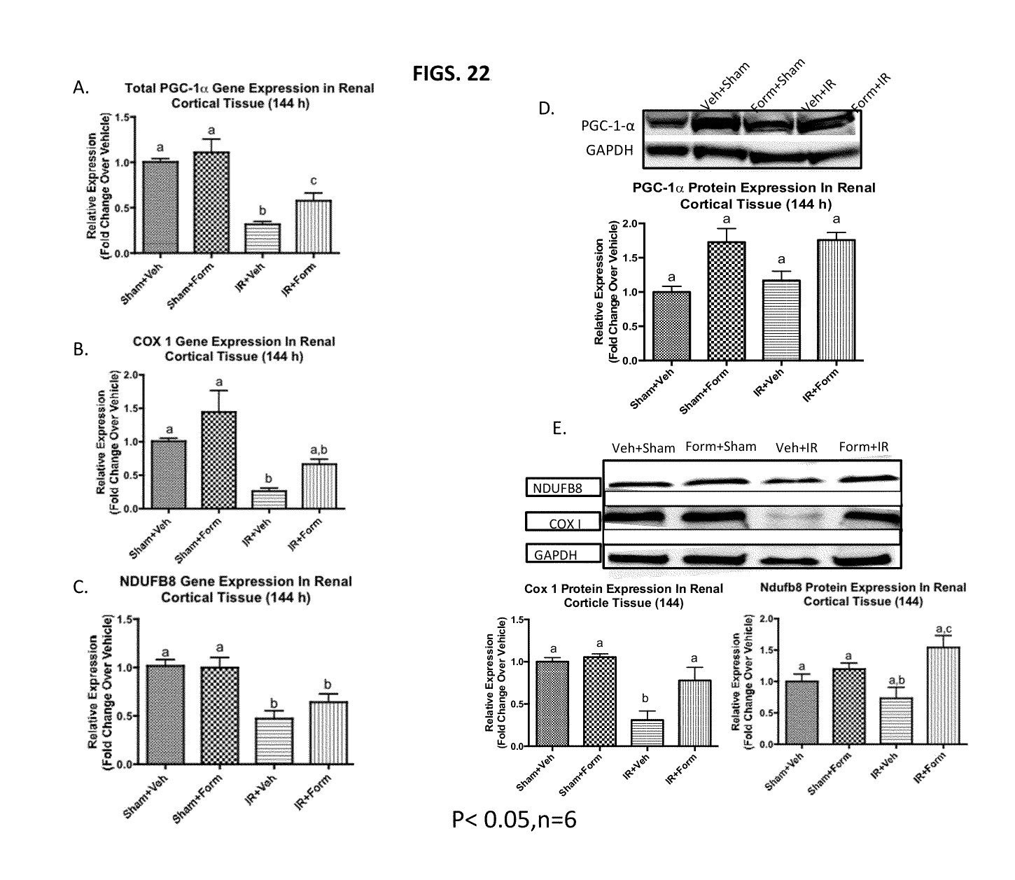

Previous in vivo and in vitro research has identified depletion of mitochondrial function and abundance in models of hypoxia correlated with dysfunctional kidney function. Furthermore, the overexpression of PGC-1α, the master regulator of mitochondrial biogenesis, has been shown to recover cellular respiration and increase ATP production after hypoxic injury. Recently, formoterol has been identified as a pharmacological agent capable of stimulating mitochondrial biogenesis in the renal cortical tissue of naïve mice. Therefore, the inventors tested if formoterol was capable of restoring the gene and protein expression of components for the mitochondrial electron transport chain and PGC-1α at the peak of kidney injury, as determined by serum creatinine (SCr), which was 24 hours. Gene transcription of total PGC-1α, COX I (nuclear-encoded), and NDUFB8 (nuclear-encoded) was significantly down regulated in the I/R vehicle group at 144 h (FIGS. 22A-C). Treatment with formoterol 24 h post I/R injury increased gene expression of total PGC-1α significantly above the I/R vehicle group, but not fully restored to control levels (FIG. 22A). Furthermore, COX I expression was restored with treatment of formoterol after I/R, but not significantly different than the I/R vehicle group (FIG. 22B). However, the formoterol I/R group did not restore NDUFB8 gene transcription to that of the control animals (FIG. 22C). Protein expression in animals subjected I/R followed by treatment with vehicle was evaluated revealing a continual suppression of NDUFB8, significant reduction in COX I, and PGC-1α expression equal to that of the control animals (FIGS. 22D-E). In contrast, treatment with formoterol after I/R restored COX I and NDBUFB8 protein expression significantly above the I/R vehicle group at 144 h (FIG. 22E). Gene transcription and protein translation did was not significantly altered in sham animals exposed to formoterol compared to the control animals.

Formoterol Restores Mitochondrial Function After I/R Injury

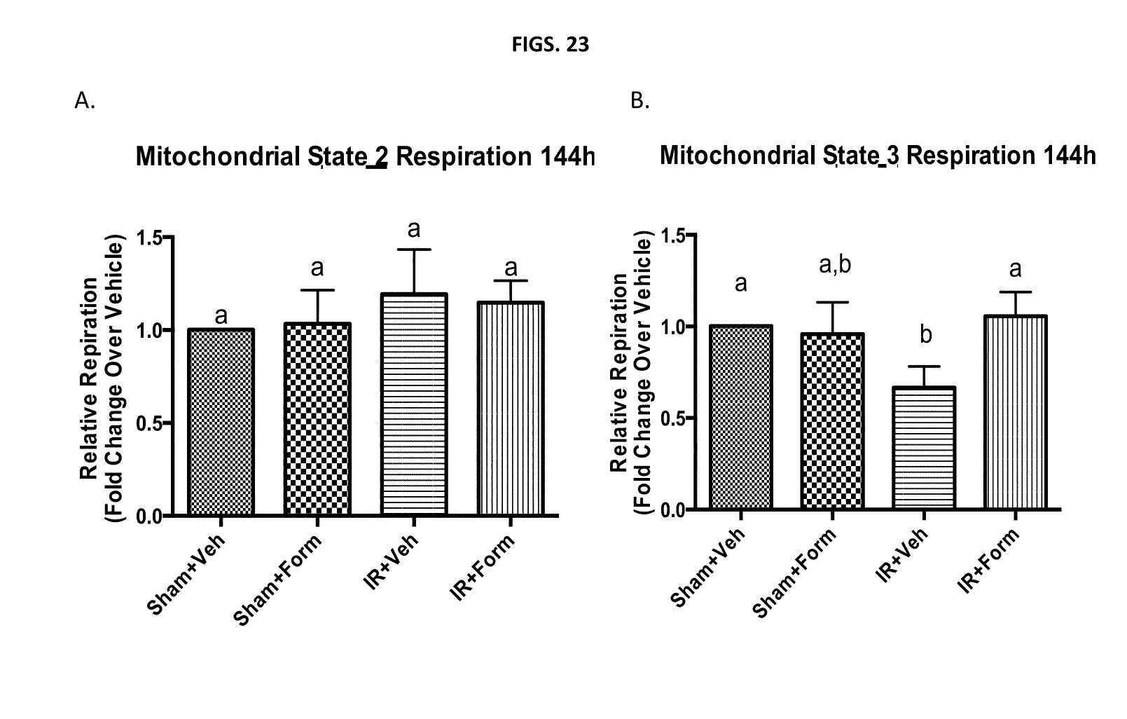

Previous models of hypoxic injury to renal proximal tubule cells have reported decreased mitochondrial function to be correlated with of tubular injury. In combination with previous findings indicative of mitochondrial biogenesis, the inventors evaluated if treatment with formoterol after I/R injury is capable of restoring mitochondrial function. Mitochondrial function was measured ex vivo by isolating renal mitochondria and determining state 4 (basal respiration) and state 3 (ADP-stimulated respiratory rate). Treatment with formoterol after I/R injury was observed to completely restore mitochondrial respiration. In contrast, treatment with vehicle after I/R was significantly decreased compared to all other groups, indicative of decreased mitochondrial function (FIGS. 23A-B).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Abrahamian, E., Fox, P. C., Naerum, L., Christensen, I. T., Thogersen, H., and Clark, R. D. (2003) Efficient generation, storage, and manipulation of fully flexible pharmacophore multiplets and their use in 3-D similarity searching, J Chem Inf Comput Sci 43, 458-468.

Agbenyega E, Morton R, Hatton P and Wareham A (1995) Effect of the beta 2-adrenergic agonist clenbuterol on the growth of fast- and slow-twitch skeletal muscle of the dystrophic (C57BL6J dy2J/dy2J) mouse. Comp Biochem Physiol C Pharmacol Toxicol Endocrinol 111:397-403.

Arany Z, Wagner B K, Ma Y, Chinsomboon J, Laznik D and Spiegelman B M (2008) Gene expression-based screening identifies microtubule inhibitors as inducers of PGC-1α and oxidative phosphorylation. Proceedings of the National Academy of Sciences 105:4721-4726.

Audet, M., and Bouvier, M. (2008) Insights into signaling from the beta2-adrenergic receptor structure, Nature chemical biology 4, 397-403.

Backman, T. W., Cao, Y., and Girke, T. (2011) ChemMine tools: an online service for analyzing and clustering small molecules, Nucleic Acids Res 39, W486-491.

Baloyannis, S. J., Costa, V., and Michmizos, D. (2004) Mitochondrial alterations in Alzheimer's disease, American journal of Alzheimer's disease and other dementias 19, 89-93.

Baloyannis S J (2006) Mitochondrial alterations in Alzheimer's disease. Journal of Alzheimer's Disease 9:119-126.

Beeson C C, Beeson G C and Schnellmann R G (2010) A high-throughput respirometric assay for mitochondrial biogenesis and toxicity. Analytical Biochemistry 404:75-81.

Birch-Machin, M. A., and Turnbull, D. M. (2001) Assaying mitochondrial respiratory complex activity in mitochondria isolated from human cells and tissues, In Methods in Cell Biology (Liza A. Pon, E. A. S., Ed.), pp 97-117, Academic Press.

Cheetham S C, Viggers J A, Butler S A, Prow M R and Heal D J (1996) [3H]Nisoxetine—A radioligand for noradrenaline reuptake sites: Correlation with inhibition of [3H]noradrenaline uptake and effect of DSP-4 lesioning and antidepressant treatments. Neuropharmacology 35:63-70.

Civitarese A E and Ravussin E (2008) Minireview: Mitochondrial Energetics and Insulin Resistance. Endocrinology 149:950-954.

de Graaf, C., and Rognan, D. (2008) Selective structure-based virtual screening for full and partial agonists of the beta2 adrenergic receptor, J Med Chem 51, 4978-4985.

Ebalunode J O, Zheng W and Tropsha A (2011) Application of QSAR and shape pharmacophore modeling approaches for targeted chemical library design. Methods in Molecular Biology 685:111-133.

Ferrick D A, Neilson A and Beeson C (2008) Advances in measuring cellular bioenergetics using extracellular flux. Drug Discovery Today 13:268-274.

Fuke S, Kubota-Sakashita M, Kasahara T, Shigeyoshi Y and Kato T (2011) Regional variation in mitochondrial DNA copy number in mouse brain. Biochimica et Biophysica Acta (BBA)—Bioenergetics 1807:270-274.

Funk J A, Odejinmi S and Schnellmann R G (2010) SRT1720 Induces Mitochondrial Biogenesis and Rescues Mitochondrial Function after Oxidant Injury in Renal Proximal Tubule Cells. Journal of Pharmacology and Experimental Therapeutics 333:593-601.

Galandrin, S., and Bouvier, M. (2006) Distinct signaling profiles of beta1 and beta2 adrenergic receptor ligands toward adenylyl cyclase and mitogen-activated protein kinase reveals the pluridimensionality of efficacy, Molecular pharmacology 70, 1575-1584.

Gerencser A A, Neilson A, Choi S W, Edman U, Yadava N, Oh R J, Ferrick D A, Nicholls D G and Brand M D (2009) Quantitative Microplate-Based Respirometry with Correction for Oxygen Diffusion. Analytical Chemistry 81:6868-6878.

Ghanouni, P., Gryczynski, Z., Steenhuis, J. J., Lee, T. W., Farrens, D. L., Lakowicz, J. R., and Kobilka, B. K. (2001) Functionally different agonists induce distinct conformations in the G protein coupling domain of the beta 2 adrenergic receptor, J Biol Chem 276, 24433-24436.

Glossop, P. A., Lane, C. A., Price, D. A., Bunnage, M. E., Lewthwaite, R. A., James, K., Brown, A. D., Yeadon, M., Perros-Huguet, C., Trevethick, M. A., Clarke, N. P., Webster, R., Jones, R. M., Burrows, J. L., Feeder, N., Taylor, S. C., and Spence, F. J. (2010) Inhalation by design: novel ultra-long-acting beta(2)-adrenoreceptor agonists for inhaled once-daily treatment of asthma and chronic obstructive pulmonary disease that utilize a sulfonamide agonist headgroup, J Med Chem 53, 6640-6652.

Goligorsky, M. S., Osborne, D., Howard, T., Hruska, K. A., and Karl, I. E. (1987) Hormonal regulation of gluconeogenesis in cultured proximal tubular cells: role of cytosolic calcium, Am J Physiol Renal Physiol 253, F802-809.

Granier, S., and Kobilka, B. (2012) A new era of GPCR structural and chemical biology, Nature chemical biology 8, 670-673.

Hagen T M, Moreau R, Suh J H and Visioli F (2002) Mitochondrial Decay in the Aging Rat Heart. Annals of the New York Academy of Sciences 959:491-507.

Hagstrom-Toft E, Enoksson S, Moberg E, Bolinder J and Amer P (1998) beta-Adrenergic regulation of lipolysis and blood flow in human skeletal muscle in vivo. Am J Physiol 275:E909-916.

Hall J, Kaumann A and Brown M (1990) Selective beta 1-adrenoceptor blockade enhances positive inotropic responses to endogenous catecholamines mediated through beta 2-adrenoceptors in human atrial myocardium. Circulation Research 66:1610-1623.

Hall, R. A., Premont, R. T., Chow, C. W., Blitzer, J. T., Pitcher, J. A., Claing, A., Stoffel, R. H., Barak, L. S., Shenolikar, S., Weinman, E. J., Grinstein, S., and Lefkowitz, R. J. (1998) The beta2-adrenergic receptor interacts with the Na+/H+-exchanger regulatory factor to control Na+/H+ exchange, Nature 392, 626-630.

Harding, S. E., and Gong, H. (2004) beta-adrenoceptor blockers as agonists: coupling of beta2-adrenoceptors to multiple G-proteins in the failing human heart, Congest Heart Fail 10, 181-185; quiz 186-187.

Higashida K, Higuchi M and Terada S (2008) Potential role of lipin-1 in exercise-induced mitochondrial biogenesis. Biochemical and Biophysical Research Communications 374:587-591.

Hirsch, T., Susin, S. A., Marzo, I., Marchetti, P., Zamzami, N., and Kroemer, G. (1998) Mitochondrial permeability transition in apoptosis and necrosis, Cell Biology and Toxicology 14, 141-145.

Horvath D (2011) Pharmacophore-Based Virtual Screening, in Chemoinformatics and Computational Chemical Biology (Bajorath J ed) pp 261-298, Humana Press.

Katritch, V., Reynolds, K. A., Cherezov, V., Hanson, M. A., Roth, C. B., Yeager, M., and Abagyan, R. (2009) Analysis of full and partial agonists binding to beta2-adrenergic receptor suggests a role of transmembrane helix V in agonist-specific conformational changes, Journal of molecular recognition: JMR 22, 307-318.

Kenakin, T., and Miller, L. J. (2010) Seven transmembrane receptors as shapeshifting proteins: the impact of allosteric modulation and functional selectivity on new drug discovery, Pharmacological reviews 62, 265-304.

Kolb P, Rosenbaum D M, Irwin J J, Fung J J, Kobilka B K and Shoichet B K (2009) Structure-based discovery of β2-adrenergic receptor ligands. Proceedings of the National Academy of Sciences 106:6843-6848.

Léger B, Koopman R, Walrand S, Gehrig S M, Murphy K T and Lynch G S (2011) Chronic formoterol administration reduces cardiac mitochondrial protein synthesis and oxidative capacity in mice. International Journal of Cardiology 146:270-272.

Little P, Safdar A, Bishop D, Tarnopolsky M A and Gibala M J (2011) An acute bout of high-intensity interval training increases the nuclear abundance of PGC-1α and activates mitochondrial biogenesis in human skeletal muscle. American Journal of Physiology—Regulatory, Integrative and Comparative Physiology 300:R1303-R1310.

Luttrell, L. M., and Kenakin, T. P. (2011) Refining efficacy: allosterism and bias in G protein-coupled receptor signaling, Methods Mol Biol 756, 3-35.

Mann D, Kent R and Cooper G (1989) Load regulation of the properties of adult feline cardiocytes: growth induction by cellular deformation. Circulation Research 64:1079-1090.

Medeiros D M (2008) Assessing mitochondria biogenesis. Methods 46:288-294.

Mitcheson J S, Hancox J C and Levi A J (1998) Cultured adult cardiac myocytes. Cardiovascular Research 39:280-300.

Miura S, Kawanaka K, Kai Y, Tamura M, Goto M, Shiuchi T, Minokoshi Y and Ezaki O (2007) An Increase in Murine Skeletal Muscle Peroxisome Proliferator-Activated Receptor-g Coactivator-1a (PGC-1a) mRNA in Response to Exercise Is Mediated by b-Adrenergic Receptor Activation. Endocrinology 148:3441-3448.

Mochizucki D (2004) Serotonin and noradrenaline reuptake inhibitors in animal models of pain. Human Psychopharmacology: Clinical and Experimental 19:S15-S19.

Nowak G and Schnellmann R G (1995) Improved culture conditions stimulate gluconeogenesis in primary cultures of renal proximal tubule cells. Am J Physiol Cell Physiol 268:C1053-1061.

Nowak G and Schnellmann R G (1996) L-ascorbic acid regulates growth and metabolism of renal cells: improvements in cell culture. Am J Physiol Cell Physiol 271:C2072-2080.

Ortega V E and Peters S P (2010) Beta-2 adrenergic agonists: focus on safety and benefits versus risks. Current Opinion in Pharmacology 10:246-253.

Pearen M, Myers S, Raichur S, Ryall J, Lynch G and Muscat G (2008) The Orphan Nuclear Receptor, NOR-1, a Target of {beta}-Adrenergic Signaling, Regulates Gene Expression that Controls Oxidative Metabolism in Skeletal Muscle. Endocrinology 149:2853-2865.

Pearen M, Ryall J, Lynch G and Muscat G (2009) Expression profiling of skeletal muscle following acute and chronic beta2-adrenergic stimulation: implications for hypertrophy, metabolism and circadian rhythm. BMC Genomics 10:448.

Pearlman D A and Charifson P S (2001) Improved Scoring of Ligand—Protein Interactions Using OWFEG Free Energy Grids. Journal of Medicinal Chemistry 44:502-511.

Penn, R. B., and Benovic, J. L. (2008) Regulation of heterotrimeric G protein signaling in airway smooth muscle, Proc Am Thorac Soc 5, 47-57.

Peterson, Y. K., Wang, X. S., Casey, P. J., and Tropsha, A. (2009) Discovery of geranylgeranyltransferase-I inhibitors with novel scaffolds by the means of quantitative structure-activity relationship modeling, virtual screening, and experimental validation, J Med Chem 52, 4210-4220.

Procopiou, P. A., Barrett, V. J., Bevan, N. J., Biggadike, K., Butchers, P. R., Coe, D. M., Conroy, R., Edney, D. D., Field, R. N., Ford, A. J., Guntrip, S. B., Looker, B. E., McLay, I. M., Monteith, M. J., Morrison, V. S., Mutch, P. J., Richards, S. A., Sasse, R., and Smith, C. E. (2009) Synthesis and structure-activity relationships of long-acting beta2 adrenergic receptor agonists incorporating arylsulfonamide groups, J Med Chem 52, 2280-2288.

Rasbach K A, Funk J A, Jayavelu T, Green P T and Schnellmann R G (2010) 5-Hydroxytryptamine Receptor Stimulation of Mitochondrial Biogenesis. Journal of Pharmacology and Experimental Therapeutics 332:632-639.

Rasbach K A and Schnellmann R G (2007) PGC-1[alpha] over-expression promotes recovery from mitochondrial dysfunction and cell injury. Biochemical and Biophysical Research Communications 355:734-739.

Rasbach K A and Schnellmann R G (2008) Isoflavones Promote Mitochondrial Biogenesis. Journal of Pharmacology and Experimental Therapeutics 325:536-543.

Rasbach, K. A., Funk, J. A., Jayavelu, T., Green, P. T., and Schnellmann, R. G. (2010) 5-Hydroxytryptamine Receptor Stimulation of Mitochondrial Biogenesis, Journal of Pharmacology and Experimental Therapeutics 332, 632-639.

Rasmussen, S. G., DeVree, B. T., Zou, Y., Kruse, A. C., Chung, K. Y., Kobilka, T. S., Thian, F. S., Chae, P. S., Pardon, E., Calinski, D., Mathiesen, J. M., Shah, S. T., Lyons, J. A., Caffrey, M., Gellman, S. H., Steyaert, J., Skiniotis, G., Weis, W. I., Sunahara, R. K., and Kobilka, B. K. (2011) Crystal structure of the beta2 adrenergic receptor-Gs protein complex, Nature 477, 549-555.

Reynolds, K. A., Katritch, V., and Abagyan, R. (2009) Identifying conformational changes of the beta(2) adrenoceptor that enable accurate prediction of ligand/receptor interactions and screening for GPCR modulators, J Comput Aided Mol Des 23, 273-288.

Rosenbaum, D. M., Cherezov, V., Hanson, M. A., Rasmussen, S. G., Thian, F. S., Kobilka, T. S., Choi, H. J., Yao, X. J., Weis, W. I., Stevens, R. C., and Kobilka, B. K. (2007) GPCR engineering yields high-resolution structural insights into beta2-adrenergic receptor function, Science 318, 1266-1273.

Sauliere, A., Bellot, M., Paris, H., Denis, C., Finana, F., Hansen, J. T., Altie, M. F., Seguelas, M. H., Pathak, A., Hansen, J. L., Senard, J. M., and Gales, C. (2012) Deciphering biased-agonism complexity reveals a new active AT1 receptor entity, Nature chemical biology 8, 622-630.

Seifert, R., Wenzel-Seifert, K., Gether, U., and Kobilka, B. K. (2001) Functional differences between full and partial agonists: evidence for ligand-specific receptor conformations, J Pharmacol Exp Ther 297, 1218-1226.

Seo A Y, Joseph A-M, Dutta D, Hwang J C Y, Aris J P and Leeuwenburgh C (2010) New insights into the role of mitochondria in aging: mitochondrial dynamics and more. Journal of Cell Science 123:2533-2542.

Springer J P, Kropp B P and Thor K B (1994) Facilitatory and inhibitory effects of selective norepinephrine reuptake inhibitors on hypogastric nerve-evoked urethral contractions in the cat: a prominent role of urethral beta-adrenergic receptors. J Urol 152:515-519.

Stallaert, W., Dorn, J. F., van der Westhuizen, E., Audet, M., and Bouvier, M. (2012) Impedance responses reveal beta(2)-adrenergic receptor signaling pluridimensionality and allow classification of ligands with distinct signaling profiles, PLoS One 7, e29420.

Suryanarayana, S., and Kobilka, B. K. (1993) Amino acid substitutions at position 312 in the seventh hydrophobic segment of the beta 2-adrenergic receptor modify ligand-binding specificity, Molecular pharmacology 44, 111-114.

Sutherland L N, Bomhof M R, Capozzi L C, Basaraba S A U and Wright D C (2009) Exercise and adrenaline increase PGC-1α mRNA expression in rat adipose tissue. The Journal of Physiology 587:1607-1617.

Tasler, S., Baumgartner, R., Aschenbrenner, A., Ammendola, A., Wolf, K., Wieber, T., Schachtner, J., Blisse, M., Quotschalla, U., and Ney, P. (2010) A vHTS approach for the identification of beta-adrenoceptor ligands, Bioorg Med Chem Lett 20, 3399-3404.

Tran M, Tam D, Bardia A, Bhasin M, Rowe G C, Kher A, Zsengeller Z K, Akhavan-Sharif M R, Khankin E V, Saintgeniez M, David S, Burstein D, Karumanchi S A, Stillman I E, Arany Z and Parikh S M (2011) PGC-1α promotes recovery after acute kidney injury during systemic inflammation in mice. The Journal of Clinical Investigation 0:0-0.

Vilardaga, J. P. (2006) Switching modes for G protein-coupled receptor activation, Nature chemical biology 2, 395-396.

Wacker, D., Fenalti, G., Brown, M. A., Katritch, V., Abagyan, R., Cherezov, V., and Stevens, R. C. (2010) Conserved binding mode of human beta2 adrenergic receptor inverse agonists and antagonist revealed by X-ray crystallography, Journal of the American Chemical Society 132, 11443-11445.

Wagner B K, Kitami T, Gilbert T J, Peck D, Ramanathan A, Schreiber S L, Golub T R and Mootha V K (2008) Large-scale chemical dissection of mitochondrial function. Nat Biotech 26:343-351.

Wallace, K. B., and Starkov, A. A. (2000) Mitochondrial Targets of Drug Toxicity, Annual Review of Pharmacology and Toxicology 40, 353-388.

Willett P (2006) Similarity-based virtual screening using 2D fingerprints. Drug Discovery Today 11:1046-1053.

Wills, L. P., Trager, R. E., Beeson, G. C., Lindsey, C. C., Peterson, Y. K., Beeson, C. C., and Schnellmann, R. G. (2012) The beta2-Adrenoceptor Agonist Formoterol Stimulates Mitochondrial Biogenesis, J Pharmacol Exp Ther 342, 106-118.

Wishart D S, Knox C, Guo A C, Shrivastava S, Hassanali M, Stothard P, Chang Z and Woolsey J (2006) DrugBank: a comprehensive resource for in silico drug discovery and exploration. Nucleic Acids Research 34:D668-D672.

Wong D T and Bymaster F P (1976) Effect of nisoxetine on uptake of catecholamines in synaptosomes isolated from discrete regions of rat brain. Biochemical Pharmacology 25:1979-1983.

Wu Z, Huang X, Feng Y, Handschin C, Feng Y, Gullicksen PS, Bare O, Labow M, Spiegelman B and Stevenson S C (2006) Transducer of regulated CREB-binding proteins (TORCs) induce PGC-1α transcription and mitochondrial biogenesis in muscle cells. Proceedings of the National Academy of Sciences 103:14379-14384.

Xiao, R. P. (2001) Beta-adrenergic signaling in the heart: dual coupling of the beta2-adrenergic receptor to G(s) and G(i) proteins, Science's STKE: signal transduction knowledge environment 2001, re15.

Zerbe R L, Rowe H, Enas G G, Wong D, Farid N and Lemberger L (1985) Clinical pharmacology of tomoxetine, a potential antidepressant. Journal of Pharmacology and Experimental Therapeutics 232:139-143.

What is claimed is:

1. A method of inducing mitochondrial biogenesis, comprising administering to a subject, identified as in need of increased mitochondrial biogenesis, a pharmacologically effective dose of a beta-2 adrenergic receptor agonist, wherein the beta-2 adrenergic receptor agonist is tomoxetine, nisoxetine, fenoterol, formoterol, or procaterol.

2. The method of claim 1, wherein a pharmacologically effective dose of tomoxetine, nisoxetine, fenoterol, or procaterol is administered to the subject.

3. The method of claim 1, wherein the compound is administered to the subject orally, intravenously, intramuscularly, intraperitoneally, topically, or via inhalation.

4. The method of claim 1, wherein the compound is comprised in a pharmaceutically acceptable formulation.

5. The method of claim 1, wherein the subject is a human.

6. The method of claim 1, wherein the subject has been identified as having deficient mitochondrial biogenesis by a muscle biopsy test, a metabolic test, or a genetic test.

7. The method of claim 1, wherein the subject has a mitochondrial injury.

8. The method of claim 7, wherein the mitochondrial injury resulted from an acute kidney injury (AKI), neurodegeneration, a heart disease, heart attack, a stroke, renal dysfunction, type 2 diabetes, a central nervous system disorder, Alzheimer's disease, Parkinson's disease, Huntington's disease, ischemia/reperfusion, trauma, a drug/toxicant-induced organ injury, chronic traumatic encephalopathy (CTE), or a traumatic brain injury (TBI).

9. The method of claim 8, wherein the mitochondrial injury resulted from an acute kidney injury.

10. The method of claim 1, wherein the subject has a mitochondrial disease.

11. The method of claim 10, wherein the mitochondrial disease is Leber's hereditary optic neuropathy, diabetes mellitus, a mental disorder or disease, Leigh's disease, mitochondrial encephalomyopathy, lactic acidosis, mitochondrial neurogastrointestinal encephalomyopathy (MNGIE), Myoclonic Epilepsy with Ragged Red Fibers (MERRF), “Neuropathy, ataxia, retinitis pigmentosa, and ptosis” (NARP), Wolff-Parkinson-White syndrome, stroke-like episodes (MELAS), or a muscle wasting disease.

12. The method of claim 10, wherein the mitochondrial disease is a muscle wasting disease.

13. A method of treating an acute kidney injury in an individual comprising administering to the individual a pharmacologically effective amount of tomoxetine, nisoxetine, fenoterol, formoterol, or procaterol.

14. The method of claim 13, wherein a pharmacologically effective amount of tomoxetine, nisoxetine, fenoterol, or procaterol is administered to the subject.

15. The method of claim 13, wherein the subject is a human.

16. A method of treating a muscle wasting or a muscle wasting disease in an individual comprising administering to the individual a pharmacologically effective amount of tomoxetine, nisoxetine, fenoterol, or procaterol.

17. The method of claim 16, wherein the muscle wasting results from cachexia, cancer cachexia, a spinal cord injury, or wasting disease.

18. The method of claim 13, wherein the subject is a human.


SEQUENCE LISTING (16 sequences)

SEQ ID NO: 1 - DNA, 18 bp, Artificial Sequence
  Synthetic primer
cgtcggctccagcattct

SEQ ID NO: 2 - DNA, 22 bp, Artificial Sequence
  Synthetic primer
tcaccagtcatttctgcctttg

SEQ ID NO: 3 - DNA, 24 bp, Artificial Sequence
  Synthetic primer
tcctgcctcactcattcacaccaa

SEQ ID NO: 4 - DNA, 24 bp, Artificial Sequence
  Synthetic primer
agtgcagcgcctccaataagatgt

SEQ ID NO: 5 - DNA, 21 bp, Artificial Sequence
  Synthetic primer
cccaagcatataagctagtac

SEQ ID NO: 6 - DNA, 25 bp, Artificial Sequence
  Synthetic primer
atataagtcatattttgggaactac

SEQ ID NO: 7 - DNA, 20 bp, Artificial Sequence
  Synthetic primer
tagaacgcaaaatcttaggg

SEQ ID NO: 8 - DNA, 20 bp, Artificial Sequence
  Synthetic primer
tgctagtgtgagtgataggg

SEQ ID NO: 9 - DNA, 24 bp, Artificial Sequence
  Synthetic primer
actgcgatggcaactgaggagtat

SEQ ID NO: 10 - DNA, 24 bp, Artificial Sequence
  Synthetic primer
accataactatacaacgccgccac

SEQ ID NO: 11 - DNA, 20 bp, Artificial Sequence
  Synthetic primer
acccaatccaaccgccttca

SEQ ID NO: 12 - DNA, 20 bp, Artificial Sequence
  Synthetic primer
ctaggaccccagaggaacgc

SEQ ID NO: 13 - DNA, 24 bp, Artificial Sequence
  Synthetic primer
aggaaatccgagctgagctgaaca

SEQ ID NO: 14 - DNA, 24 bp, Artificial Sequence
  Synthetic primer
gcaagaaggcgacacatcgaacaa

SEQ ID NO: 15 - DNA, 21 bp, Artificial Sequence
  Synthetic primer
ctctctgtcgattacggcaag

SEQ ID NO: 16 - DNA, 21 bp, Artificial Sequence
  Synthetic primer
tggtgaggatggagttgtagg